(19) United States
(12) Patent Application Publication
(10) Pub. No.: US 2023/0144384 A1
(43) Pub. Date: May 11, 2023

(54) PARA-PHENYLENEDIAMINE BASES WITH CATIONIC HETEROCYCLES, AND THE USE OF SAME FOR OXIDATION DYEING KERATIN FIBRES

(71) Applicant: L'OREAL, Paris (FR)

(72) Inventors: Aziz FADLI, AULNAY-SOUS-BOIS (FR); Zhibo LIU, AULNAY-SOUS-BOIS (FR)

(21) Appl. No.: 16/648,197

(22) PCT Filed: Sep. 27, 2018
(86) PCT No.: PCT/EP2018/076269

§ 371 Date: Jun. 3, 2020

(30) Foreign Application Priority Data
Sep. 29, 2017 (FR) 1759087

(51) Int. Cl. A61K 8/49; A61Q 5/10; A61K 8/41; A61K 8/34; A61K 8/22; C07D 233/64; C07D 295/135

(52) U.S. Cl. CPC A61K 8/4946; A61Q 5/10; A61K 8/4926; A61K 8/415; A61K 8/411; A61K 8/34; A61K 8/22; C07D 233/64; C07D 295/135

(57) ABSTRACT

The invention relates to primary para-phenylenediamine compounds substituted with an aliphatic chain comprising a cationic heterocyclic group according to formula (I), as well as the organic or mineral acid or base addition salts thereof, optical isomers, geometric isomers, tautomers, mesomers and/or solvates thereof, such as hydrates. Formula (I), wherein: ALK is a linear or branched, optionally substituted, alkylene chain comprising 3 to 8 carbon atoms; HET⁺ is a saturated or unsaturated, aromatic or non-aromatic cationic heterocyclic group, optionally substituted, comprising 5 to 0 links, optionally substituted, and comprising one or more ammonium groups; and An⁻, present or absent, is a mineral or organic anionic counterion ensuring the electroneutrality of the molecule. The present invention also relates to a composition comprising one or more of these previously defined compounds, in a medium suitable for dyeing. The present invention lastly relates to a dyeing device consisting of a first compartment which contains said composition, and a second compartment containing one or more oxidising agents.

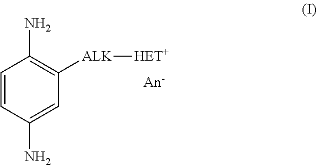

### CROSS REFERENCE TO RELATED APPLICATIONS

This is a national stage application of PCT/EP2018/076269, filed internationally on Sep. 27, 2018, which claims priority to French Application No. 1759087, filed Sep. 29, 2017, both of which are incorporated by reference herein in their entireties.

A subject matter of the present invention is para-phenylenediamine compounds substituted by an aliphatic chain comprising a specific cationic heterocyclic group.

The present invention is used in the field of the dyeing of keratin fibers and more particularly the dyeing of keratin fibers, especially human keratin fibers such as the hair.

It is already known from the prior art that para-phenylenediamine bases play an important role in the process of hair dyeing. They are colorless or weakly colored oxidation dye precursors which, in the presence of oxidizing compounds, are transformed into colored compounds.

By combining an oxidation dye precursor with oxidizing compounds and dyeing couplers, a broad palette rich in color is obtained.

“Permanent” dyeing is characterized by the use of dye precursors in the presence of oxidizing compounds. In order to be considered as efficient dyeing, the latter needs to satisfy certain criteria. It must make it possible to obtain shades in the desired intensity with differences in coloring, between the tip and the root of one and the same lock (also known as selectivity), which are as small as possible.

The dyes must also be resistant over time and must not become degraded in the presence of external agents, such as washing, light, bad weather, rubbing and perspiration. Application DE 102010038714 discloses compounds of primary para-phenylenediamine type substituted by a group comprising a quaternary ammonium in order to color keratin fibers. Application EP 2 791 109 also discloses para-phenylenediamine bases. Although these oxidation bases make it possible to obtain a wide range of colors, combinations thereof with conventional couplers sometimes lack homogeneity and chromaticity and the selectivities are often considerable. French patent application FR 3044899 also describes para-phenylenediamines with an alkylene chain comprising an aromatic or non-aromatic heterocycle. However, the dyeing results obtained are not always satisfactory, especially in terms of color buildup, of selectivity, of chromaticity, of intensity and/or of persistence, in particular with respect to successive shampoo washes, or of resistance to light or to perspiration.

There is thus a real need to provide colorings which exhibit better dyeing properties, in particular in terms of chromaticity, of selectivity, of strength and of fastness, and which are also capable of resulting in a wide range of colors.

The present invention addresses these problems. A subject matter of the present invention is new compounds of primary para-phenylenediamine type substituted by aliphatic chains comprising a cationic heterocyclic group of formula (I) as defined below. These compounds make it possible to obtain better dyeing properties, and in particular a better solubility, color buildup, chromaticity, fastness and selectivity. They also afford access to a wide range of light, natural and dark colors.

The invention also relates to the use of one or more para-phenylenediamine compounds substituted by aliphatic chains comprising a cationic heterocyclic group of formula (I) as defined below, in the presence of one or more oxidizing agents, for dyeing keratin fibers, in particular human keratin fibers, such as the hair.

The present invention additionally relates to a composition for dyeing keratin fibers, in particular human keratin fibers, such as the hair, comprising, in a medium appropriate for dyeing, one or more para-phenylenediamine compounds substituted by aliphatic chains comprising a cationic heterocyclic group of formula (I) as defined below.

In particular, the invention relates to the use of said composition for dyeing keratin fibers, in particular human keratin fibers, such as the hair.

The invention also relates to a method for dyeing keratin fibers, in particular human keratin fibers, such as the hair, in which said dyeing composition according to the invention is applied to said fibers in the presence of one or more oxidizing agents for a time sufficient to obtain the desired coloring, after which the resulting fibers are rinsed, optionally washed with a shampoo, rinsed again and dried or left to dry.

Another subject matter of the present invention concerns a dyeing multicompartment device or kit comprising a first compartment containing a dyeing composition as described above and a second compartment containing one or more oxidizing agents. The multicompartment device is thus appropriate for the implementation of the dyeing method according to the invention.

I—Para-Phenylenediamine Compound with Cationic Heterocyclic Group

A subject matter of the present invention is thus new compounds of primary para-phenylenediamine type substituted by an aliphatic chain comprising at least one cationic group of formula (I), the addition salts thereof with organic or inorganic acids or bases, optical isomers thereof, geometric isomers thereof, tautomers thereof, mesomers thereof and/or solvates thereof such as hydrates:

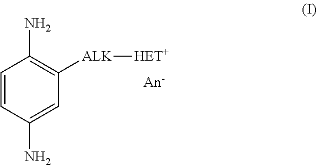

in which formula (I):
ALK represents a linear or branched alkylene chain comprising from 3 to 8 carbon atoms which is optionally substituted;
HET⁺ represents a saturated or unsaturated, aromatic or non-aromatic cationic heterocyclic group, which is optionally substituted, comprising from 5 to 10 ring members, and comprising one or more ammonium groups; and
An⁻, which is present or absent, represents an inorganic or organic anionic counterion, ensuring the electrical neutrality of the molecule.

In the context of the presentation, unless otherwise mentioned: The term “alkyl” denotes a saturated or unsaturated, linear or branched hydrocarbon group comprising from 1 to 6 carbon atoms; preferably, the alkyl group is saturated, in particular, the alkyl group is a saturated C₁-C₄ group, more particularly the alkyl group is a saturated, linear C₁-C₄ group, such as a methyl or ethyl group; The term “alkoxy” denotes an alkyl-oxy group with alkyl as defined above, preferably methoxy or ethoxy;

The term “alkylthio” denotes an alkyl-S-group with alkyl as defined above, preferably methylthio or ethylthio;

The term “alkylene” corresponds to a linear or branched divalent C₃-C₈ hydrocarbon group of general formula CₙH₂ₙ with 3≤n≤8, in particular a C₃-C₆ group; preferably a C₃-C₄ group, such as propylene;

When the aryl or heterocyclic radical is “optionally substituted”, this implies that said radical can be substituted by one or more radicals chosen from the following radicals: a) hydroxyl, b) C₁-C₂ alkoxy or alkyl, c) C₂-C₄ (poly)hydroxyalkoxy or (poly)hydroxyalkyl, d) amino substituted by one or two identical or different C₁-C₄ alkyl radicals optionally bearing at least one hydroxyl group or it being possible for the two radicals to form, with the nitrogen atom to which they are attached, a heterocycle comprising from 5 to 7 ring members, preferably 5 or 6 ring members, said heterocycle formed being saturated or unsaturated and optionally comprising another heteroatom identical to or different from nitrogen; e) a halogen atom; f) acylamino (—NR—C(O)—R′) in which the R radical is a hydrogen atom or a C₁-C₄ alkyl radical optionally bearing at least one hydroxyl group and the R′ radical is a C₁-C₂ alkyl radical, such as a methyl radical; g) carbamoyl ((R)2N—C(O)—) in which the R radicals, which are identical or different, represent a hydrogen atom or a C₁-C₄ alkyl radical optionally bearing at least one hydroxyl group; h) alkylsulfonylamino (R′—S(O)₂—N(R)—) in which the R radical represents a hydrogen atom or a C₁-C₄ alkyl radical optionally bearing at least one hydroxyl group and the R′ radical represents a C₁-C₄ alkyl radical or a phenyl radical; i) aminosulfonyl ((R)₂N—S(O)₂—) in which the R radicals, which are identical or different, represent a hydrogen atom or a C₁-C₄ alkyl radical optionally bearing at least one hydroxyl group; j) carboxy in the acid or salified=carboxylate C(O)O⁻ form (preferably with an alkali metal or a substituted or unsubstituted ammonium); k) cyano l) nitro or nitroso; m) polyhalo(C₁-C₄)alkyl, such as trifluoromethyl; n) —N⁺(Rₑ)(R_{f})(R_{g}), An^{'} with Rₑ, R_{f} and R_{g} independently denoting a (C₁-C₆)alkyl radical or a hydroxy(C₁-C₆)alkyl radical and An⁻ as defined above; and o) oxo;

An “aryl” radical represents a monocyclic or polycyclic, fused or non-fused carbon-based group comprising from 6 to 12 carbon atoms and at least one ring of which is aromatic; preferentially, the aryl radical is a phenyl, biphenyl, naphthyl, indenyl, anthracenyl or tetrahydronaphthyl; more preferentially, the aryl radical in the invention represents a phenyl group;

A “heterocyclic radical” is an aromatic or non-aromatic, monocyclic or bicyclic, fused or non-fused radical which may contain one or more unsaturations, that contains from 5 to 10 ring members, that comprises from 1 to 4 heteroatoms chosen from a nitrogen, oxygen and sulfur atom, preferably chosen from an oxygen atom or a nitrogen atom; as an example of a preferred heterocyclic radical of the invention, mention may be made of the radicals chosen from pyrrolyl, pyrrolidinyl, (benzo)imidazolyl, (benzo)pyrazolinyl, (benzo)oxazolinyl, (benzo)thiazolinyl, indolinyl, (benzo)triazolinyl, piperidinyl, piperazinyl, morpholinyl, thiomorpholinyl and 4-aza-1-azoniabicyclo [2.2.2]octane;

A “cationic heterocyclic radical” is a heterocyclic radical as defined above, which comprises one or more =N⁺(Rₑ)—, An⁻ or —N⁺(Rₑ)(R_{f})—, An⁻ ammonium group(s);and/or said —N⁺(Rₑ)(R_{f})(R_{g}), An⁻ ammonium group(s) being borne by one or more substituents of the heterocycle or else said —N⁺(Rₑ)(R_{f})(R_{g}), An⁻ ammonium group(s) being one or more substituents of the heterocycle, with Rₑ, R_{f}, R_{g} independently denoting a (C₁-C₆)alkyl radical or a hydroxy(C₁-C₆)alkyl radical, preferably a (C₁-C₄)alkyl radical; said cationic heterocyclic radical being particularly chosen from:
i) cationic heterocycles, at least one of the ring members of which is an =N⁺(Rₑ)-, An⁻ or —N⁺(Rₑ)(R_{f})—, An⁻ ammonium radical; and more particularly the cationic heterocycles are chosen from pyrrolium, pyrrolinium, pyrrolidinium, (benzo)imidazolium, (benzo)pyrazolinium, (benzo)oxazolinium, (benzo)thiazolinium, indolinium, (benzo)triazolinium, piperidinium, piperazinium, piperazin-1,4-dium, morpholinium, thiomorpholinium and 4-aza-1-azoniabicyclo[2.2.2]octane, and 1,4-diazoniabicyclo[2.2.2]octane, optionally substituted by one or more (C₁-C₄)alkyl or hydroxy(C₁-C₄)alkyl groups, in particular optionally substituted by one or more (C₁-C₄)alkyl groups such as a methyl or ethyl group;
ii) heterocycles substituted by a radical bearing an —N⁺(Rₑ)(R_{f})(R_{g}), An⁻, cationic group, preferably a saturated heterocycle such as a pyrrolidinyl heterocycle, substituted by a (C₁-C₄)alkyl radical, said alkyl radical being substituted by an —N⁺(Rₑ)(R_{f})(R_{g}), An⁻ radical, such as a trimethylammonium radical; and
iii) heterocycles substituted by an —N⁺(Rₑ)(R_{f})(R_{g}), An cationic radical, preferably a saturated heterocycle such as a pyrrolidinyl heterocycle, substituted by an —N⁺(Rₑ)(R_{f})(R_{g}), An⁻ radical, such as a trimethylammonium radical;

An “aromatic heterocyclic radical” represents a monocyclic or bicyclic, fused or non-fused group which is optionally cationic, comprising from 5 to 10 ring members, from 1 to 4 heteroatoms chosen from a nitrogen, oxygen and sulfur atom, preferably chosen from a nitrogen atom or an oxygen atom, and at least one ring of which is aromatic; preferentially;

An “aromatic heterocyclic” radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridinyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenooxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthylyl;

The expression “optionally substituted” attributed to the alkyl or alkylene radical implies that said alkyl or alkylene radical can be substituted by one or more halogen atoms or radicals chosen from the following radicals: i) hydroxyl, ii) C₁-C₄ alkoxy, iii) acylamino, iv) amino optionally substituted by one or two identical or different C₁-C₄ alkyl radicals, it being possible for said alkyl radicals to form, with the nitrogen atom which bears them, a heterocycle comprising from 5 to 7 ring members, said heterocycle optionally comprising another heteroatom different or not from nitrogen and optionally being substituted, and v) carboxy or carboxylate;

When the alkoxy group is optionally substituted, this implies that the alkyl group of the alkyl-oxy is optionally substituted as defined above;

The term “anionic counterion” denotes an anion or a mixture of anions resulting from salt(s) of organic or inorganic acid(s) counterbalancing the cationic charge of the dye; more particularly, the anionic counterion is chosen from i) halides, such as chloride or bromide; ii) nitrates; iii) sulfonates, including C₁-C₆ alkyl sulfonates: Alk-S(O)₂O⁻, such as methylsulfonate or mesylate and ethylsulfonate; iv) arylsulfonates: Ar—S(O)₂O⁻, such as benzenesulfonate and toluenesulfonate or tosylate; v) citrate; vi) succinate; vii) tartrate; viii) lactate; ix) alkyl sulfates: Alk-O—S(O)O⁻, such as methyl sulfate and ethyl sulfate; x) aryl sulfates: Ar—O—S(O)O⁻, such as benzene sulfate and toluene sulfate; xi) alkoxy sulfates: Alk-O—S(O)₂O⁻, such as methoxy sulfate and ethoxy sulfate; xii) aryloxy sulfates: Ar—O—S(O)₂O⁻, xiii) phosphates O═P(OH)₂—O⁻, O═P(O⁻)₂—OH, O═P(O⁻)₃, HO—[P(O)(O—)]_{w}—P(O)(O—)₂ with w being an integer; xiv) acetate; xv) triflate; xvi) borates, such as tetrafluoroborate, and xvii) disulfate (O═)₂S(O⁻)₂ or SO₄²⁻ and monosulfate HSO₄⁻;

The anionic counterion resulting from an organic or inorganic acid salt ensures the electrical neutrality of the molecule, and it is understood that, when the anion comprises several anionic charges, then the same anion can serve for the electrical neutrality of several cationic groups in the same molecule or else can serve for the electrical neutrality of several molecules;

When An⁻ is “present”, if HET⁺ comprises several cationic ammonium groups, then it can have several An⁻ groups, which are identical or different, to ensure the electrical neutrality of the molecule;

When An⁻ is “absent” this implies that an anionic group is present on the molecule to compensate for the cationic charge of the HET⁺ group, for example a C(O)O⁻ carboxylate group;

The term “organic or inorganic acid addition salt” is understood more particularly to mean salts chosen from a salt derived i) from hydrochloric acid HCl, ii) from hydrobromic acid HBr, iii) from sulfuric acid H₂SO₄, iv) from alkylsulfonic acids: Alk-S(O)₂OH, such as methylsulfonic acid and ethylsulfonic acid; v) from arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH, such as methoxysulfinic acid and ethoxysulfinic acid; xi) from aryloxysulfinic acids, such as tolueneoxysulfinic acid and phenoxysulfinic acid; xii) from phosphoric acid H₃PO₄; xiii) from acetic acid CH₃C(O)OH; xiv) from triflic acid CF₃SO₃H; and xv) from tetrafluoroboric acid HBF₄; preferably, the acid salts according to the invention are salts of inorganic acids, such as hydrochloric acid salts.

According to one particular embodiment, the compounds of formula (I) are such that HET⁺ is chosen from the formulae (A) to (P) below:

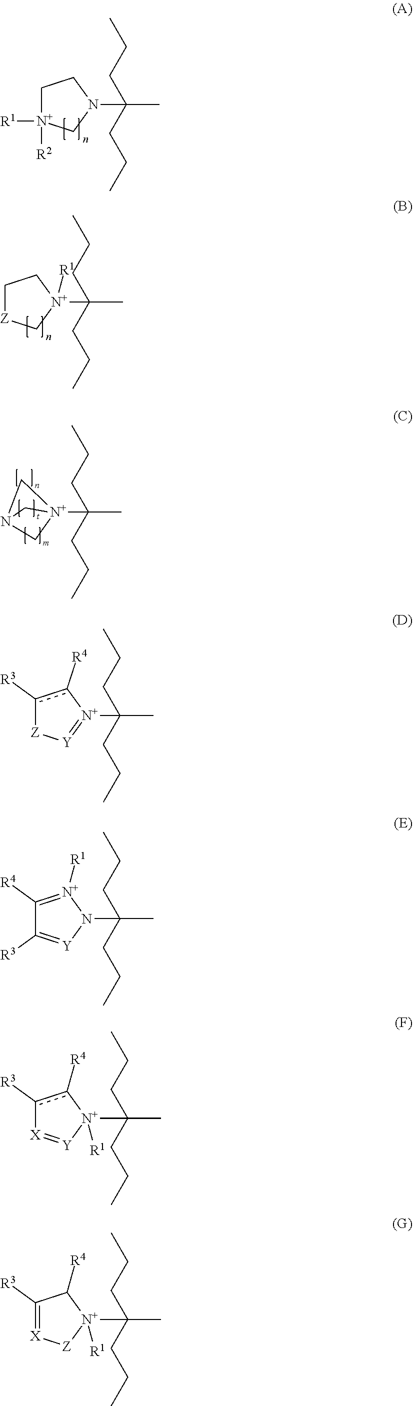

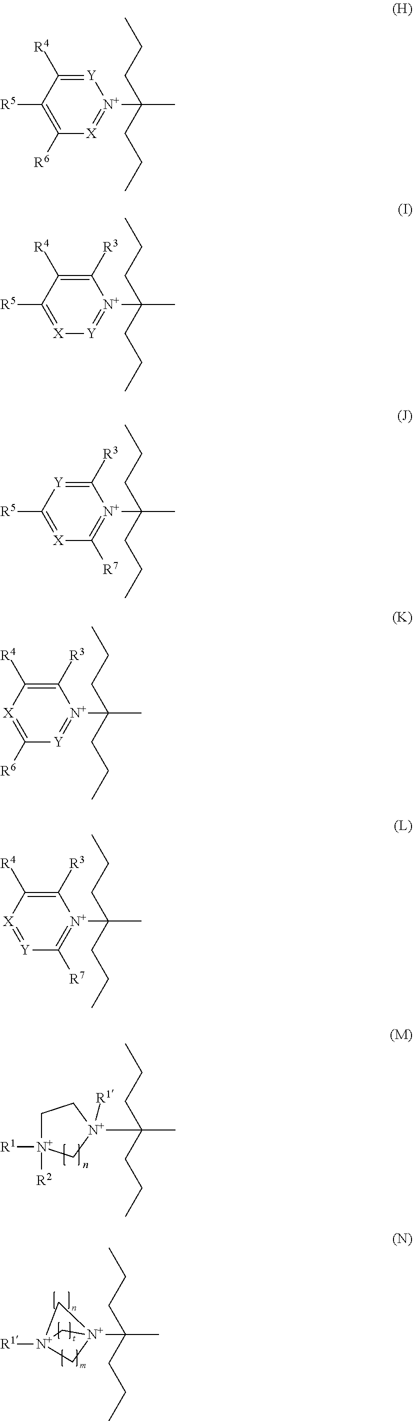

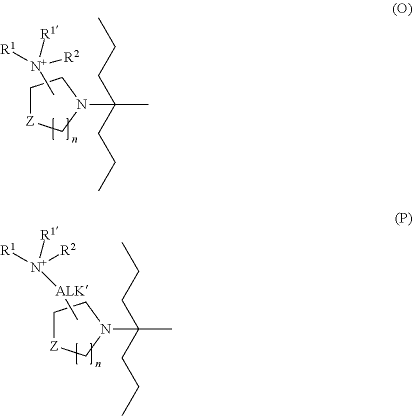

with
n being equal to 1, 2 or 3, preferably 1 or 2;
m and t, which are identical or different, being equal to 1, 2 or 3, preferably 2;
R¹, R^{1′} and R², which are identical or different, represent a (C₁-C₆)alkyl group, or a hydroxy(C₁-C₆)alkyl group, preferably a (C₁-C₄)alkyl group such as a methyl group;
R³, R⁴, R⁵, R⁶ and R⁷, which are identical or different, represent a hydrogen atom, a halogen atom or a group chosen from (C₁-C₆)alkyl, (C₁-C₆)alkoxy, (C₁-C₆)alkylthio, (di)(C₁-C₆)(alkyl)amino, nitro(so), R⁸—(Z′)ₚ—C(Z″)—(Z″′)_{q}— with p and q, which are identical or different, being equal to 0 or 1, Z′, Z″ and Z″′, which are identical or different, representing an oxygen atom, a sulfur atom or an N(R⁹) group with R⁸ and R⁹, which are identical or different, representing a hydrogen atom, a (C₁-C₄)alkyl group or a hydroxy(C₁-C₄)alkyl group; or else two contiguous chosen from R³, R⁴, R⁵, R⁶, R⁸ and R⁹, form, together with the carbon atoms which bear them, a benzo group; preferentially R³, R⁴, R⁵, R⁶ and R⁷ represent a hydrogen atom or two contiguous groups chosen from R³ and R⁴, or R′ and R⁵, or R⁵ and R⁶ form, together with the carbon atoms which bear them, a benzo group; more preferentially R³, R⁴, R⁵, R⁶ and R⁷ represent a hydrogen atom; -1 X and Y, which are identical or different, represent a nitrogen atom, or a group chosen from C(R⁸) with R⁸ as defined previously or R⁸ represents an (C₁-C₄)alkylcarbonyl group such as an acyl group, preferably R⁸ represents a hydrogen atom;
Z representing an oxygen atom, a sulfur atom, a C(R⁸)(R⁹) group or N(R⁹) group with R⁸ and R⁹ as defined previously, preferably R⁸ and R⁹ represent a hydrogen atom;
ALK′ represents a linear or branched (C₁-C₄)alkylene group, which is optionally substituted, preferably which is unsubstituted and/or linear;

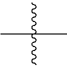

represents the part of the group linked to the rest of the molecule; and
- - - - - representing a single bond or double bond, preferably a double bond;
it being understood that for (O) and (P) the (R¹)(R′₁)(R²)N⁺— and (R¹)(R′₁)(R²)N⁺-ALK′-ammonium groups respectively may be borne by Z when Z represents a C(R⁸)(R⁹) group, in which case one of the two R⁸ or R⁹ groups is absent.

According to one advantageous embodiment of the invention, the HET⁺ group is chosen from (A) and (B) with n being equal to 1 or 2, and Z preferably representing an oxygen atom or a C(R⁸)(R⁹) or N(R⁹) group, more preferentially Z representing an oxygen atom, methylene CH₂ or N—R⁹′ with R⁹′ representing a (C₁-C₄)alkyl group such as a methyl group.

According to one preferred variant of the invention HET⁺ represents the (A₁) group

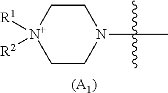

with R¹ and R², as defined previously, which are identical or different, preferably identical, representing in particular a (C₁-C₄)alkyl group such as a methyl group.

According to another preferred variant of the invention, HET⁺ represents the group chosen from (B₁), (B₂), (B₃), (B₄) and (B₅) below, and more particularly the group chosen from (B₁), (B₂), (B₄) and (B₅)

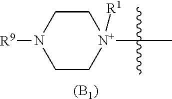

with R¹ and R⁹, as defined previously, which are identical or different, preferably identical, representing in particular a (C₁-C₄)alkyl group such as a methyl group;

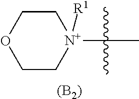

with R¹ as defined previously preferably representing a (C₁-C₄)alkyl group such as a methyl group;

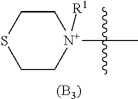

with R¹ as defined previously preferably representing a (C₁-C₄)alkyl group such as a methyl group;

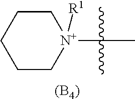

with R¹ as defined previously preferably representing a (C₁-C₄)alkyl group such as a methyl group;

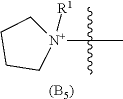

with R¹ as defined previously preferably representing a (C₁-C₄)alkyl group such as a methyl group;

According to another advantageous embodiment of the invention, HET⁺ represents the (C) group with n, m and t, which are identical or different, being equal to 1, 2 or 3, preferably n, m and t are identical, particularly n, m and t are equal to 2. According to one preferred variant of the invention HET⁺ represents the (Ci) group below:

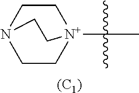

According to another advantageous embodiment of the invention, HET⁺ represents the (D) group with Y representing a group chosen from C(R⁸) with R⁸ as defined above, preferably R⁸ represents a hydrogen atom; Z preferably representing an oxygen atom or a C(R⁸)(R⁹) or N(R⁹) group, more preferentially Z=CH₂ or N—R⁹′ with R⁹′ representing a (C₁-C₄)alkyl group such as a methyl or ethyl group; R³ and R⁴ representing a hydrogen atom, a (C₁-C₄)alkyl group such as a methyl group, or else R³ and R⁴ form, together with the carbon atoms which bear them, a benzo group; preferably R3 and R₄ represent a hydrogen atom or a (C₁-C₄)alkyl group such as a methyl group; and - - - - - representing a double bond.

According to another advantageous embodiment of the invention HET⁺ represents (D) and more particularly HET⁺ represents the imidazolium group (D₁) or the mesomeric form thereof (D′1):

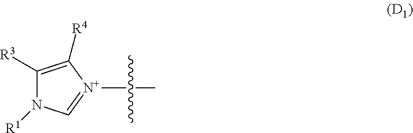

with R³, R⁴ and R¹ as defined previously, preferably R³ and R⁴ represent a hydrogen atom and R¹ represents a (C₁-C₄)alkyl group such as a methyl or ethyl group.

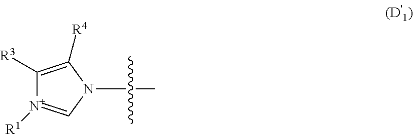

According to another advantageous embodiment of the invention HET⁺ represents the (E) group with Y representing a C(R⁸) group with R⁸ as defined above, preferably R⁸ represents a hydrogen atom; R³ and R⁴ representing a hydrogen atom, a (C₁-C₄)alkyl group such as a methyl group; preferably R3 and R4 represent a hydrogen atom. More particularly HET⁺ represents the pyrazolium group (E₁):

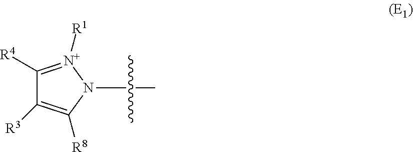

with R³, R⁴, R⁸ and R¹ as defined above, preferably R³, R⁴ and R⁸ represent a hydrogen atom and R¹ represents a (C₁-C₄)alkyl group such as a methyl or ethyl group.

According to another advantageous embodiment of the invention HET⁺ represents the (F) group with X and Y, which are identical or different, representing a C(R⁸) group with R⁸ as defined above. More particularly HET⁺ represents the pyrrolium group (F₁):

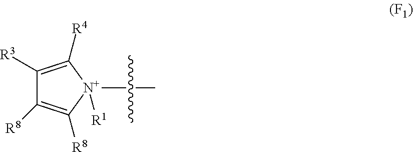

with R³, R⁴, R⁸ and R¹ as defined above, preferably R³, R⁴ and R⁸ represent a hydrogen atom and R¹ represents a (C₁-C₄)alkyl group such as a methyl or ethyl group.

According to another advantageous embodiment of the invention HET⁺ represents the (G) group with X representing a C(R⁸) group with R⁸ as defined above, and Z representing C(R⁸)(R⁹) with R¹, R³, R⁴, R⁸ and R⁹ as defined above. More particularly HET⁺ represents the pyrrolinium group (G₁):

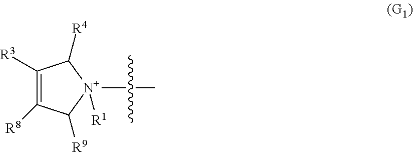

with R¹, R³, R⁴, R⁸ and R⁹ as defined above, preferably R³, R⁴, R⁸ and R⁹ represent a hydrogen atom and R¹ represents a (C₁-C₄)alkyl group such as a methyl or ethyl group.

According to another advantageous embodiment of the invention HET⁺ represents the (O) group with n being equal to 1 or 2, preferably 1, and Z as defined above, preferably representing an oxygen atom or a C(R⁸)(R⁹) group, more preferentially Z representing a methylene group, and R¹, R′¹ and R² representing a (C₁-C₄)alkyl group such as a methyl group. More particularly HET⁺ represents the pyrrolidinyl group (O₁):

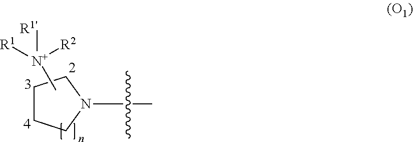

with R¹ and R², as defined above, preferably R¹, R′¹, and R², represent a (C₁-C₄)alkyl group such as a methyl group, n is equal to 1 or 2, preferably n=1, it being understood that the (R¹)(R′₁)(R²)N⁺— ammonium group is in position 2, 3 or 4, preferably in position 3 or 4.

According to another advantageous embodiment of the invention HET⁺ represents the (P) group with n being equal to 1 or 2, preferably 1, and Z as defined above, preferably representing an oxygen atom or a C(R⁸)(R⁹) group, more preferentially Z representing a methylene group, and R¹, R′¹ and R² representing a (C₁-C₄)alkyl group such as a methyl group and ALK′ as defined above, preferably methylene, ethylene, or propylene. More particularly HET⁺ represents the pyrrolidinyl group (P₁):

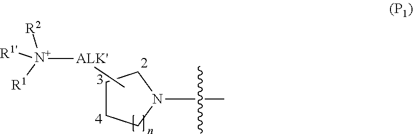

with R¹, R′¹, and R², represent a (C₁-C₄)alkyl group such as a methyl group, n is equal to 1 or 2, preferably =1, ALK′ represents a linear (C₁-C₄)alkylene group such as methylene, ethylene, or propylene, it being understood that the (R¹)(R′₁)(R²)N⁺-ALK-group is in position 2, 3 or 4, preferably in position 3 or 4.

According to a specific embodiment of the invention, ALK represents a linear unsubstituted C₃-C₆ alkylene chain, preferably a linear unsubstituted C₃-C₅ alkylene chain, more particularly a linear unsubstituted C₃-C₄ alkylene chain, more preferably still a linear unsubstituted C₃ alkylene chain, such as a propylene —(CH₂)₃— chain. Preferentially, the compounds of formula (I) are chosen from the following compounds 1 to 13, the addition salts thereof with organic or inorganic acids, optical isomers thereof, geometric isomers thereof, tautomers thereof, mesomers thereof and/or solvates thereof such as hydrates:

| | | |
|---|---|---|
| Compound 1 | 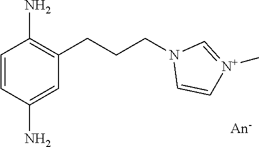 [SMILES: C[n+]1ccn(CCCc2cc(N)ccc2N)c1] | 1-[3-(2,5- diaminophenyl)propyl]-3- methyl-1H-imidazol-3-ium salt |
| Compound 2 | 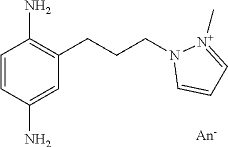 [SMILES: C[n+]1cccn1CCCc1cc(N)ccc1N] | 1-[3-(2,5- diaminophenyl)propyl]-2- methyl-1H-pyrazol-1-ium salt |
| Compound 3 | 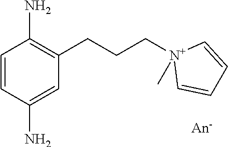 [SMILES: C[N+]1(CCCc2cc(N)ccc2N)C=CC=C1] | 1-[3-(2,5- diaminophenyl)propyl]-1- methyl-1H-pyrrol-1-ium salt |
| Compound 4 | 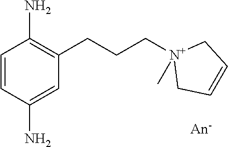 [SMILES: C[N+]1(CCCc2cc(N)ccc2N)CC=CC1] | 1-[3-(2,5- diaminophenyl)propyl]-1- methyl-2,5-dehydro-1H-pyrrol- 2-ium salt |
| Compound 5 | 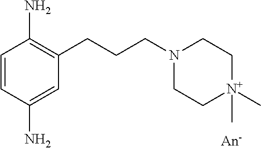 [SMILES: C[N+]1(C)CCN(CCCc2cc(N)ccc2N)CC1] | 4-[3-(2,5- diaminophenyl)propyl]-1,1- dimethylpiperazin-1-ium salt |
| Compound 6 | 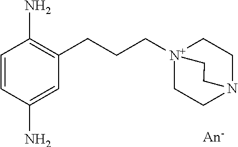 [SMILES: Nc1ccc(N)c(CCC[N+]23CCN(CC2)CC3)c1] | 1-[3-(2,5- diaminophenyl)propyl]-4-aza-1- azoniabicyclo[2.2.2]octane salt |
| Compound 7 | 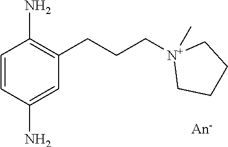 [SMILES: C[N+]1(CCCc2cc(N)ccc2N)CCCC1] | 1-[3-(2,5- diaminophenyl)propyl]-1- methylpyrrolidinium salt |
| Compound 8 | 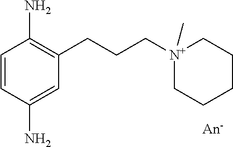 [SMILES: C[N+]1(CCCc2cc(N)ccc2N)CCCCC1] | 1-[3-(2,5- diaminophenyl)propyl]-1- methylpiperidinium salt |
| Compound 9 | 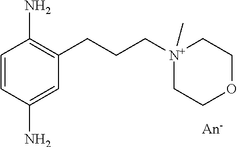 [SMILES: C[N+]1(CCCc2cc(N)ccc2N)CCOCC1] | 4-[3-(2,5- diaminophenyl)propyl]-4- methylmorpholin-4-ium salt |
| Compound 10 | 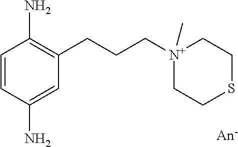 [SMILES: C[N+]1(CCCc2cc(N)ccc2N)CCSCC1] | 4-[3-(2,5- diaminophenyl)propyl]-4- methylthiomorpholin-4-ium salt |
| Compound 11 | 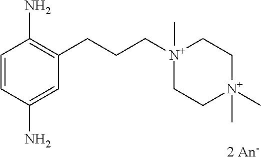 [SMILES: C[N+]1(C)CC[N+](C)(CCCc2cc(N)ccc2N)CC1] | 4-[3-(2,5- diaminophenyl)propyl]-4,4- trimethylpiperazin-1,4-diium salt |
| Compound 12 | 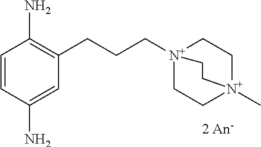 [SMILES: C[N+]12CC[N+](CCCc3cc(N)ccc3N)(CC1)CC2] | 1-[3-(2,5- diaminophenyl)propyl]-4-aza-1- methyl-1- azoniabicyclo[2.2.2]octane salt |
| Compound 13 | 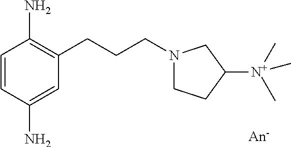 [SMILES: C[N+](C)(C)C1CCN(CCCc2cc(N)ccc2N)C1] | 1-[3-(2,5- diaminophenyl)propyl]-1- pyrrolidinyl-3- trimethylammonium salt |

with An⁻, which is identical or different, representing, as above, an anionic counterion, in particular halides, preferably chloride.

Preferably, the cationic para-phenylenediamines (I) according to the invention are chosen from compounds 1 and 5, and also the mixtures thereof.

Another subject matter of the invention is a process for the preparation of cationic para-phenylenediamines of formula (I) as defined below according to the following scheme:

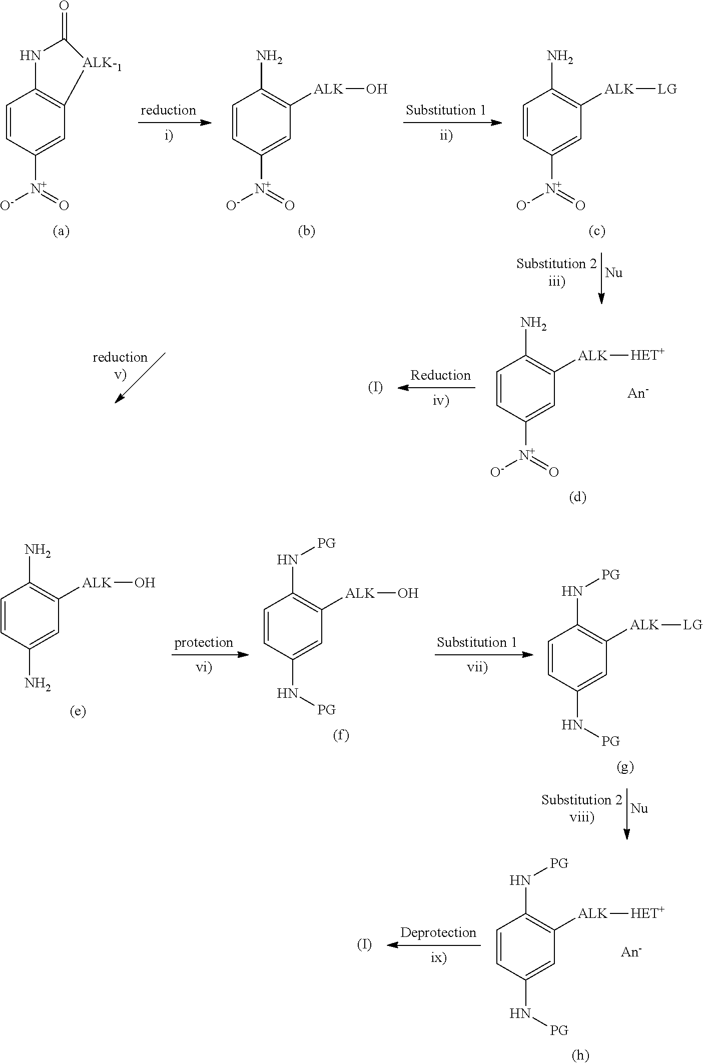

in which scheme Nu represents a Nucleophilic group comprising the cationic group HET⁺ as defined above, ALK is as defined above, ALK₋₁ represents a linear or branched alkylene chain comprising from 2 to 7 carbon atoms which is optionally substituted, LG represents a leaving group, such as halogen, tosyl or triflate, and PG represents a protecting group resistant to the reaction conditions throughout the synthesis up to the deprotection step; which process consists:

either, in a first step i), in reducing the bicyclic amido derivative (a) in order to result in the 4-nitroaniline compound substituted in the a position with respect to the amino by a hydroxypropyl group (b); preferably, this step is performed in a polar protic or aprotic organic solvent such as a (C₁-C₆)alkanol, in particular methanol or tetrahydrofuran (THF), in the presence of a reducing agent chosen in particular from borohydrides of an alkaline agent, such as NaBH4; then, in a second step ii), in transforming the hydroxyl group of the compound (b) into a leaving group LG such as halogen, triflate, tosylate, mesylate, via a (C₁-C₆)alkylsulfonyl halide such as methanesulfonyl chloride, step ii) performed in particular in a polar or non-polar protic solvent such as THF, preferably in the presence of an alkaline agent such as N,N-diisopropylethylamine (DIPEA); then, in a third step iii), in substituting the leaving group LG with a cationic group HET⁺ as defined above, preferably in alkaline medium; then, in a fourth step iv), in reducing the nitro compound (d) in particular by catalytic hydrogenation preferably with palladium, nickel, zinc, iron or tin, preferably on graphite, such as Pd(II)/C, in order to result in the compound of formula (I) of the invention;

or, in a second step v), in reducing the 4-nitroaniline derivative substituted in the a position with respect to the amino by a hydroxypropyl group (b), preferably by catalytic hydrogenation, in order to result in the 1,4-phenylenediamine compound substituted in the a position with respect to the amino by a hydroxypropyl group (e), then, in a third step vi), in protecting the amino groups with a protecting group, in particular via a reagent such as R—C(Yₐ)—Yₐ—C(Yₐ)—R′, with R and R′, which are identical or different, representing a (C₁-C₆)alkyl group and Yₐ, which is identical or different, representing an oxygen or sulfur atom; preferably, said reagent is acetic anhydride; followed by steps vii) and viii) of substitutions 1 and 2 under the same conditions as during steps ii) and iii), in order to result in the compounds (c) and (d) respectively, and then the compound (h) is deprotected, in particular in an organic solvent, preferably a polar protic organic solvent, more preferentially in the presence of one or more inorganic or organic acids, more preferentially inorganic acids, such as hydrochloric acid.

The invention also relates to the use of one or more cationic para-phenylenediamine compounds of formula (I) as defined above, preferably in the presence of one or more oxidizing agents, for dyeing keratin fibers, in particular human keratin fibers, such as the hair.

II—Dyeing Composition

The present invention additionally relates to a composition for dyeing keratin fibers, in particular human keratin fibers, such as the hair, comprising, in a medium appropriate for dyeing, one or more para-phenylenediamine compounds substituted by aliphatic chains comprising a cationic group of formula (I) as defined above.

Preferably, the dyeing composition comprises one or more cationic para-phenylenediamine compounds of formula (I), in particular those for which ALK denotes a propylene —(CH2)3— chain, and more particularly the dyeing composition according to the invention contains at least a compound chosen from compounds 1 and 5 and the mixtures thereof.

The cationic para-phenylenediamines as defined above can be present in the composition according to the invention in a content ranging from 0.1% to 20% by weight, preferably in a content ranging from 0.1% to 5% by weight, with respect to the total weight of the dyeing composition.

According to a specific embodiment of the invention, the composition of the invention and the method additionally comprise or employ one or more coupling agents or “couplers” chosen from those conventionally used in the coloring of keratin fibers.

Preferably, the coupler(s) which are conventionally used for the dyeing of keratin fibers are chosen from meta-phenylenediamines, meta-aminophenols, meta-diphenols, naphthalene couplers, heterocyclic couplers and the addition salts thereof.

More particularly, the coupler(s) used in the invention are chosen from 1,3-dihydroxybenzene, 1,3-dihydroxy-2-methylbenzene, 4-chloro-1,3-dihydroxybenzene, 2,4-diam ino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole and 6-methylpyrazolo[1,5-a]benzimidazole, 2-methyl-5-aminophenol, 5-N-(β-hydroxyethyl)amino-2-methylphenol, 3-am inophenol, 3-amino-2-chloro-6-methylphenol, the corresponding addition salts with an acid and the corresponding mixtures; most preferentially, 2,4-diamino-1-β-hydroxyethyloxy)benzene.

In general, the addition salts of oxidation bases and of coupling agents which can be used in the context of the invention are chosen in particular from the addition salts with an acid, such as the hydrochlorides, hydrobromides, sulfates, citrates, succinates, tartrates, lactates, tosylates, benzenesulfonates, phosphates and acetates.

According to a specific embodiment, the dyeing composition of the invention further comprises one or more additional oxidation bases conventionally used for the dyeing of keratin fibers, other than the compounds of formula (I).

The additional oxidation base(s) each advantageously represent(s) from 0.001% to 10% by weight, with respect to the total weight of the composition, and preferably from 0.005% to 5% by weight, with respect to the total weight of the composition and of the ready-for-use composition.

The coupling agent(s), if it is (they are) present, each advantageously represent(s) from 0.001% to 10% by weight, with respect to the total weight of the composition, and preferably from 0.005% to 5% by weight, with respect to the total weight of the composition and of the ready-for-use composition.

According to a specific embodiment of the invention, the dye(s) of formula (I) as defined above are in the presence of additional oxidation bases. Preferably, these additional bases are chosen from para-phenylenediamines other than (I), bis(phenyl)alkylenediamines, ortho-aminophenols and heterocyclic bases, and the corresponding addition salts.

The para-phenylenediamines other than the compounds of formula (I) which may be mentioned include in particular para-phenylenediamine (PPD), para-toluenediamine (PTD), 2-chloro-1,4-phenylenediamine, 2,3-dimethyl-1,4-phenylenediamine, 2,6-dimethyl-1,4-phenylenediamine, 2,6-diethyl-1,4-phenylenediamine, 2,5-dimethyl-1,4-phenylenediamine, N, N-dimethyl-1,4-phenylenediamine, N, N-diethyl-para-phenylenediamine, N, N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-1,4-phenylenediamine, 2-methoxymethyl-1,4-phenylenediamine, 2-fluoro-1,4-phenylenediamine, 2-isopropyl-1,4-phenylenediamine, N-(β-hydroxypropyl)-para-phenylenediamine, 2-hydroxymethyl-1,4-phenylenediamine, N,N-dimethyl-3-methyl-1,4-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-1,4-phenylenediamine, 2-β-acetylaminoethyloxy-1,4-phenylenediamine, N-(β-methoxyethyl)-para-phenylenediamine, 4-aminophenylpyrrolidine, 2-thienyl-1,4-phenylenediamine, 2-β-hydroxyethylamino-5-aminotoluene and 3-hydroxy-1-(4′-aminophenyl)pyrrolidine, and the corresponding addition salts with an acid. Preferentially, the oxidation base(s) of the invention are chosen from PPD, PTD, N,N-bis(β-hydroxyethyl)-para-phenylenediamine, 2-β-hydroxyethyl-1,4-phenylenediamine, 2-methoxyoxyethyl-1,4-phenylenediamine and 2-isopropyloxyethyl-1,4-phenylenediamine.

Preference is in particular given, among the abovementioned para-phenylenediamines, to para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid.

The bis(phenyl)alkylenediamines which can be mentioned include, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenylpethylenediamine, N,N′-bis(4-aminophenyl)tetramethylenediamine, N,N′-bis(β-hydroxyethyl)-N,N′-bis(4-aminophenyl)tetramethylenediamine, N, N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

The ortho-aminophenols which can be mentioned include, for example, 2-aminophenol, 2-amino-5-methylphenol, 2-amino-6-methylphenol and 5-acetamido-2-aminophenol, and the corresponding addition salts.

The heterocyclic bases which can be mentioned include, for example, pyridine, pyrimidine and pyrazole derivatives.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include 2-acetylaminopyrazolo[1,5-a]pyrid-3-ylamine, 2-morpholin-4-ylpyrazolo[1,5-a]pyrid-3-ylamine, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 2-[(3-aminopyrazolo[1,5-a]pyrid-5-yl)(2-hydroxyethyl)-amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine, 2-(4-dimethylpiperazinium-1-yl)-3-aminopyrazolo[1,5-a]pyridine, and the corresponding addition salts.

More particularly, the additional oxidation bases in the present invention are chosen from 3-aminopyrazolo[1,5-a]pyridines and preferably substituted on the carbon atom 2 by:

a) a (di)(C₁-C₆)(alkyl)amino group, said alkyl group possibly being substituted with at least one hydroxyl, amino or imidazolium group;

b) an optionally cationic heterocycloalkyl group containing from 5 to 7 ring members and from 1 to 3 heteroatoms which is optionally substituted by one or more (C₁-C₆)alkyl groups, such as a di(C₁-C₄)alkylpiperazinium or -imidazolium group; or

c) a (C₁-C₆)alkoxy group optionally substituted by one or more hydroxyl groups, such as a β-hydroxyalkoxy group, and the corresponding addition salts.

According to a specific embodiment of the invention, the additional oxidation bases are chosen from pyrazoles and preferably 4,5-diaminopyrazoles optionally substituted in 1 and/or 3 position by a (C₁-C₁₀)alkyl, (poly)hydroxy(C₁-C₁₀)alkyl, (di)(C₁-C₄)(alkyl)amino(C₁-C₁₀)alkyl or heterocyclo(C₁-C₁₀)alkyl group:

In particular, the pyrazoles are chosen from the compounds of following formula (Va) below:

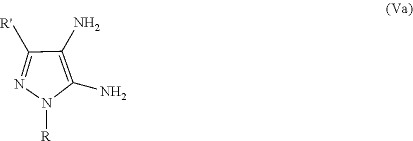

and also the addition salts thereof with organic or mineral acids, the tautomers thereof, and the solvates thereof such as hydrates: in which formula (Va):
R represents a (C₁-C₁₀)alkyl group optionally substituted by one or more hydroxyl groups,
R′ represents a hydrogen atom or a (C₁-C₄)alkyl group optionally substituted by a hydroxyl or amino group; preferably, R′ represents a (C₁-C₄)alkyl group, such as a methyl group.

Preferably, the heterocyclic bases are chosen from the bases of formula (Va) in which R′ represents a hydrogen atom or methyl and R represents an ethyl, β-hydroxyethyl or n-hexyl group. The heterocyclic bases are chosen from the following compounds (VIIa1) to (VIIa4) and also the salts thereof with organic or inorganic acids and the solvates thereof, such as hydrates:

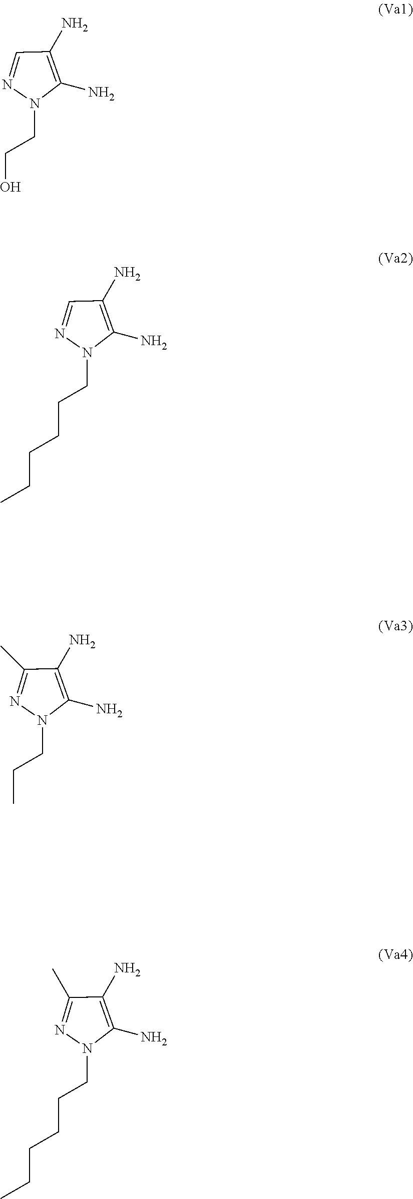

The pyrimidine derivatives which may be mentioned include the compounds described, for example, in the patents DE 2359399, JP 88-169571, JP 05-63124 and EP 0 770 375 or the patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

In general, the addition salts of the additional oxidation bases and of the couplers which can be used in the context of the invention are chosen in particular from the addition salts with an acid, such as hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, aqueous ammonia, amines or alkanolamines.

The dyeing composition in accordance with the invention can additionally contain one or more direct dyes which can in particular be chosen from nitrobenzene dyes, azo direct dyes and methine direct dyes. These direct dyes can be of nonionic, anionic or cationic in nature.

The medium appropriate for dyeing, also known as dyeing vehicle, is cosmetically acceptable, i.e. generally comprises water or a mixture of water and of one or more solvents, such as, for example, lower C₁-C₄ alkanols, such as ethanol and isopropanol, polyols, such as propylene glycol, dipropylene glycol or glycerol, and polyol ethers, such as dipropylene glycol monomethyl ether.

The solvent(s) is (are) generally present in proportions which can be between 1% and 40% by weight approximately, with respect to the total weight of the dyeing composition, and more preferentially still between 3% and 30% by weight approximately.

The dyeing composition in accordance with the invention can also contain various adjuvants conventionally used in hair dyeing compositions, such as anionic, cationic, nonionic, amphoteric or zwitterionic surfactants or their mixtures, anionic, cationic, nonionic, amphoteric or zwitterionic polymers or their mixtures, inorganic or organic thickening agents and in particular anionic, cationic, nonionic and amphoteric polymeric associative thickeners, antioxidants, penetrating agents, sequestering agents, fragrances, buffers, dispersing agents, conditioning agents, such as, for example, volatile or non-volatile and modified or unmodified silicones, film-forming agents, ceramides, preservatives or opacifying agents.

The above adjuvants are generally present in an amount for each of them of between 0.01% and 20% by weight, with respect to the weight of the composition.

Of course, a person skilled in the art will take care to choose this or these optional additional compound(s) so that the advantageous properties intrinsically attached to the oxidation dyeing composition in accordance with the invention are not, or not substantially, detrimentally affected by the envisaged addition(s).

The pH of the dyeing composition in accordance with the invention is generally between 3 and 12 approximately and preferably between 5 and 11 approximately. It can be adjusted to the desired value by means of acidifying or basifying agents generally used in the dyeing of keratin fibers, or alternatively using conventional buffer systems.

Mention may be made, among the acidifying agents, by way of example, of inorganic or organic acids, such as hydrochloric acid, orthophosphoric acid, sulfuric acid, carboxylic acids, such as acetic acid, tartaric acid, citric acid or lactic acid, or sulfonic acids.

Mention may be made, among the basifying agents, by way of example, of aqueous ammonia, alkali metal carbonates, (C₁-C₆)alkanolamines, such as mono-, di- and triethanolamines and also their derivatives, sodium hydroxide, potassium hydroxide and the compounds of following formula (II):

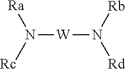

in which W is a (C₁-C₁₀)alkylene group, such as a propylene group optionally substituted by one or more hydroxyl groups; Ra, Rb, Rc and Rd, which are identical or different, represent a hydrogen atom or a C₁-C₄ alkyl or C₁-C₄ hydroxyalkyl radical.

The composition according to the invention can comprise one or more oxidizing agents. According to a preferred embodiment, the composition which comprises one or more compounds of formula (I) as defined above additionally comprises one or more oxidizing agents.

The term “oxidizing agent” is understood to mean chemical oxidizing agents other than atmospheric oxygen.

The oxidizing agents are those conventionally used for the oxidation dyeing of keratin fibers and are in particular chosen from hydrogen peroxide, urea hydrogen peroxide, alkali metal bromates, persalts, such as perborates and persulfates, peracids and oxidase enzymes, among which may be mentioned peroxidases, 2-electron oxidoreductases, such as uricases, and 4-electron oxygenases, such as laccases. Hydrogen peroxide is particularly preferred.

The dyeing composition, with or without oxidizing agent, according to the invention can be provided in various forms, such as in the form of liquids, creams or gels, or in any other form suitable for carrying out dyeing of keratin fibers, especially human keratin fibers and in particular the hair.

It can result from the mixing, at the time of use, of several compositions.

In particular, it results from the mixing of at least two compositions, one comprising one or more oxidation bases chosen from the compounds of formula (I) and/or their addition salts with an acid, optionally one or more additional oxidation bases other than the compounds of formula (I) or their salts, and optionally one or more couplers, and a second composition comprising one or more oxidizing agents as described above.

The present patent application relates to a method for dyeing keratin fibers, in particular human keratin fibers, such as the hair, in which said dyeing composition according to the invention is applied to said fibers in the presence of one or more oxidizing agents for a time sufficient to obtain the desired coloring, after which the resulting fibers are rinsed, optionally washed with a shampoo, rinsed again and dried or left to dry.

The color can be revealed at acidic, neutral or alkaline pH and the oxidizing agent can be added to the composition of the invention only at the time of use or it can be employed starting from an oxidizing composition containing it, applied simultaneously with or sequentially to the composition of the invention.

According to a specific embodiment of the invention, the composition which comprises the compound(s) of formula (I) as defined above is devoid of oxidizing agent and is mixed, preferably at the time of use, with a composition containing, in a medium appropriate for the dyeing, one or more oxidizing agents as defined above, these oxidizing agents being present in an amount sufficient to develop a coloration. The mixture obtained is subsequently applied to the keratin fibers.

In accordance with this specific embodiment, a ready-for-use composition is available which is a mixture of a composition according to the invention with one or more oxidizing agents.

After a leave-on time of the composition according to the invention of 3 to 50 minutes approximately, preferably 5 to 30 minutes approximately, the keratin fibers can be rinsed, washed with a shampoo, rinsed again and then dried.

The oxidizing composition may also contain various adjuvants conventionally used in compositions for dyeing the hair and as defined above.

The pH of the oxidizing composition including the oxidizing agent is such that, after mixing with the dyeing composition, the pH of the resulting composition applied to the keratin fibers preferably varies between 3 and 12 approximately and more preferably still between 5 and 11. It can be adjusted to the desired value by means of acidifying or basifying agents generally used in the dyeing of keratin fibers and as defined above.

Another subject matter of the invention is a dyeing multicompartment device or “kit” in which a first compartment contains the dyeing composition devoid of oxidizing agent of the present invention defined above, said dyeing composition devoid of oxidizing agent comprising one or more oxidation bases chosen from the compounds of formula (I) or addition salts thereof with an acid, and a second compartment contains one or more oxidizing agents.

These devices can be equipped with a means which makes it possible to dispense the desired mixture over the hair, such as the devices described in the patent FR-2 586 913 on behalf of the applicant company. The examples that follow serve to illustrate the invention without, however, being limiting in nature.

### EXAMPLES

### Examples of Syntheses

### Example 1

### synthesis of 1-[3-(2,5-diaminophenyl)propyl]-3-methyl-1H-imidazol-3-ium chloride dihydrochloride

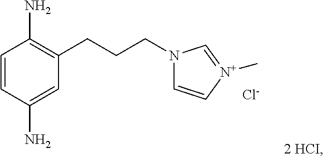 [SMILES: C[n+]1ccn(CCCc2cc(N)ccc2N)c1 ; [Cl-]]

### Example 2

### synthesis of 4-[3-(2,5-diaminophenyl)propyl]-1,1-dimethylpiperazin-1-ium chloride dihydrochloride

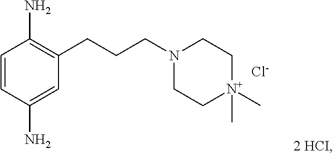 [SMILES: C[N+]1(C)CCN(CCCc2cc(N)ccc2N)CC1 ; [Cl-]]

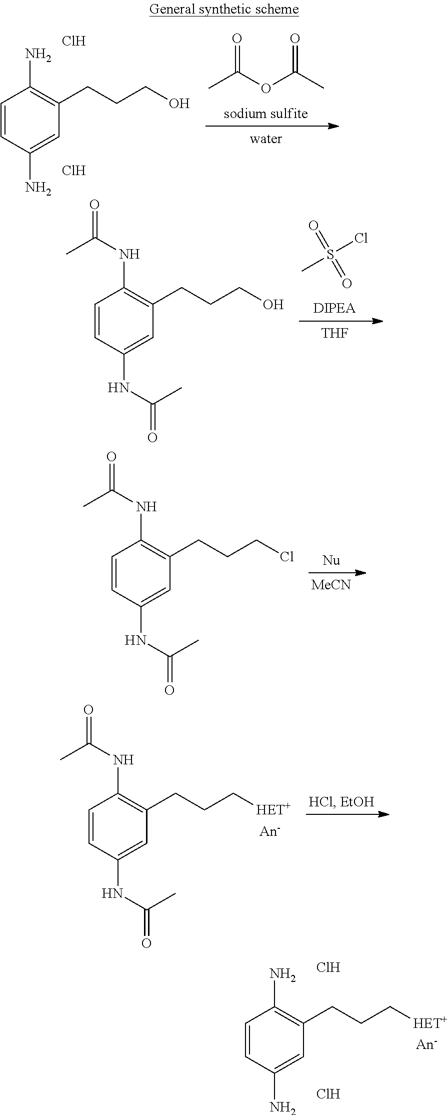

Preparation of the intermediate: synthesis of N,N′-[2-(3-chloropropyl)benzene-1,4-diyl]diacetamide

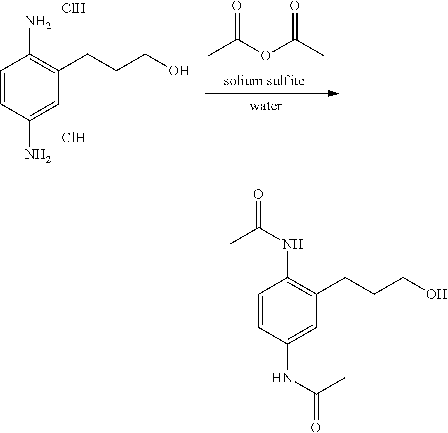

The synthesis of N,N′-[2-(3-hydroxypropyl)benzene-1,4-diyl]diacetamide may be carried out according to two processes starting from 3-(2,5-diaminophenyl)propan-1-ol dihydrochloride.

1^{st} process: 7.45 g of 3-(2,5-diaminophenyl)propan-1-ol dihydrochloride in 45 ml of water are introduced into a 250 ml three-necked flask equipped with a thermometer and a magnetic stirring bar, and then a solution of 6.22 g of sodium sulfite in 20 ml of water is added with stirring. 10 ml of acetic anhydride are then added dropwise to the reaction medium while cooling and then the mixture is left stirring at ambient temperature for 3 h. TLC (AcOEt/MeOH: 90/10) monitoring shows that the reaction is complete. The reaction medium is transferred into a flask containing 50 ml of n-BuOH. After stirring and separation of the two phases, the aqueous phase is extracted twice with n-BuOH. The combined organic phases are washed once with water and then once with saturated sodium chloride solution. After having been dried over anhydrous magnesium sulfate, the organic phase is concentrated under reduced pressure and a white powder is obtained which is then washed with hot ethyl acetate in order to result in the expected product N,N′-[2-(3-hydroxypropyl)benzene-1,4-diyl]diacetamide in the form of a white powder.

2^{nd} process: 100.62 g of 3-(2,5-diaminophenyl)propan-1-ol dihydrochloride in 500 ml of water are introduced into a 1 liter round-bottomed flask equipped with a dropping

funnel and a magnetic stirring bar, and then 58.0 g of sodium sulfite in 30 ml of water are added with stirring. 84 ml of acetic anhydride are added dropwise over 30 min while cooling and then the mixture is left stirring at ambient temperature for 3 h. TLC (AcOEt/MeOH: 90/10) monitoring shows that the reaction is not complete. A further 57 ml of acetic anhydride are added in three fractions and the mixture is left stirring at ambient temperature for 3 h after each addition. It is checked by TLC monitoring that the reaction is complete. The reaction medium (suspension) is filtered then the filtrate is transferred to a separating funnel and is extracted several times with AcOEt. The combined organic phases are concentrated under reduced pressure and the expected product is obtained in the form of a white powder after having been washed and dried.

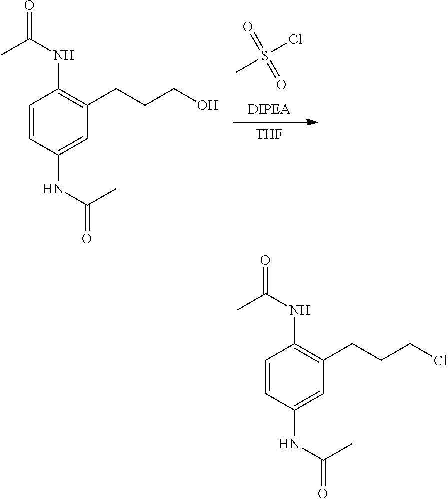

The synthesis of the intermediate N,N′-[2-(3-chloropropyl)benzene-1,4-diyl]diacetamide is carried out starting from N,N′-[2-(3-hydroxypropyl)benzene-1,4-diyl]diacetamide.

60 g of N,N′-[2-(3-hydroxypropyl)benzene-1,4-diyl]diacetamide in 1150 ml of anhydrous THF are introduced into a 2 liter three-necked flask equipped with a thermometer, a dropping funnel and a magnetic stirring bar and a milky suspension is obtained. 50 ml of N,N-diisopropylethylamine in 30 ml of anhydrous THF are then added with stirring. 23 ml of methanesulfonyl chloride are added dropwise over 1 hour while cooling, and then the mixture is left stirring at ambient temperature before being refluxed until TLC (AcOEt/MeOH: 80/20) monitoring shows that the reaction is complete. The reaction medium is transferred into a mixture of water and AcOEt. The insoluble matter is filtered and, after washing and drying, a portion of the product is thus isolated in the form of a white powder. The filtrate is evaporated under reduced pressure and the residue is purified by flash chromatography on silica gel (eluent: 5/95˜10/90 MeOH/AcOEt). After removing the solvent, the expected product is also obtained in the form of a white powder.

### Example 1

### Synthesis of 1-[3-(2,5-diaminophenyl)propyl]-3-methyl-1H-imidazol-3-ium chloride dihydrochloride

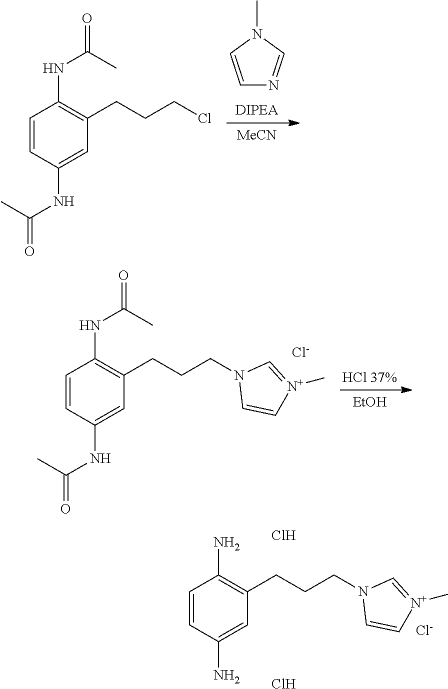

25 g of N,N′-[2-(3-chloropropyl)benzene-1,4-diyl]diacetamide in 450 ml of acetonitrile are introduced into a 1 liter three-necked flask equipped with a thermometer and a magnetic stirring bar and then 31 ml of methylimidazole are added with stirring to the suspension and the mixture is refluxed for 30 h. Since the reaction is incomplete (TLC monitoring), 10 ml of methylimidazole are added to the reaction medium and then the mixture is left stirring for an additional 10 h. The reaction medium is cooled to room temperature. The precipitate formed is filtered and washed with AcOEt, then dried in a desiccator. 1-{3-[2,5-Bis(acetylamino)phenyl]propyl}-3-methyl-1 H-imidazol-3-ium chloride is obtained in the form of a white powder.

27.0 g of 1-{3-[2,5-bis(acetylamino)phenyl]propyl}-3-methyl-1 H-imidazol-3-ium chloride obtained previously in 250 ml of ethanol are introduced into a 1 liter three-necked flask equipped with a thermometer and a magnetic stirring bar, and 150 ml of 37% hydrochloric acid are carefully added. After refluxing for 4 h, the reaction is medium is evaporated under reduced pressure (rotary evaporator). The residue is taken up in isopropanol and again evaporated to dryness with a rotary evaporator. The residue is dissolved in a minimum of methanol and then this solution is poured dropwise into isopropanol. The precipitate obtained is filtered off and then washed with ethanol before being dried in a desiccator. 1-[3-(2,5-Diaminophenyl)propyl]-3-methyl-1H-imidazol-3-ium chloride dihydrochloride is obtained in the form of a beige powder.

### Example 2

### Synthesis of 4-[3-(2,5-diaminophenyl)propyl]-1,1-dimethylpiperazin-1-ium chloride dihydrochloride

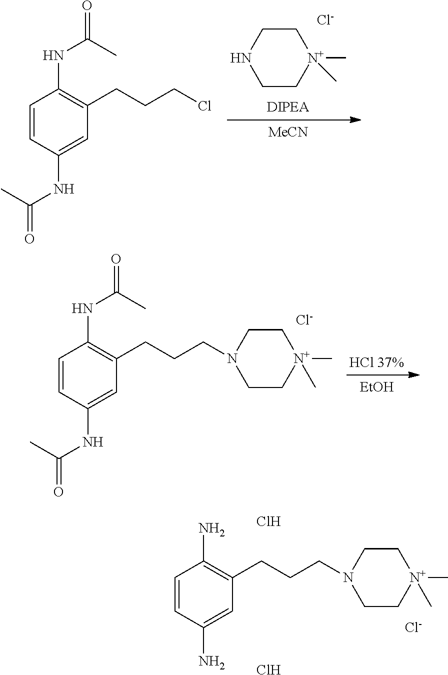

2.5 g of N,N′-[2-(3-chloropropyl)benzene-1,4-diyl]diacetamide in 30 ml of acetonitrile are introduced into a 100 ml three-necked flask equipped with a thermometer and a magnetic stirring bar and then 1.92 ml of 1,1-dimethylpiperazin-1-ium and 3.6 ml of N,N-diisopropylethylamine are added. After refluxing for 40 h, the reaction medium is filtered and the precipitate is washed with acetonitrile. The precipitate is taken up in acetonitrile and the mixture is stirred at 50° C. for 1 h, and then a hot filtration is carried out, before drying the precipitate in a desiccator. 4-{3-[2,5-Bis(acetylamino)phenyl]propyl}-1,1-dimethylpiperazin-1-ium chloride is obtained in the form of a beige powder. 2.5 g of the product obtained previously in 15 ml of ethanol and, carefully, 15 ml of 37% hydrochloric acid are introduced into a 100 ml three-necked flask equipped with a thermometer and a magnetic stirring bar. After refluxing for 5 h, the reaction medium is evaporated with a rotary evaporator. The residue is taken up in isopropanol and evaporated to dryness with a rotary evaporator. The residue is taken up in ethanol and after 30 min of stirring at 50° C., it is filtered then placed in a desiccator. 4-[3-(2,5-diaminophenyl)propyl]-1,1-dimethylpiperazin-1-ium chloride dihydrochloride is obtained in the form of a beige powder.

### DYEING EXAMPLE

### Example 1

### Dye evaluation

Dyeing vehicle:

| Compositions | Amount | |
|---|---|---|
| Compound of formula (I) | 0.05 | mmol |
| Oxidation coupler | 0.05 | mmol |
| Water | 7.0 | ml |
| Ethanol | 2.0 | ml |
| 20% aqueous ammonia solution | 1.0 | ml |
| 6-Volume aqueous hydrogen peroxide solution | 1.0 | ml |

The following oxidation couplers were used:

| Oxidation coupler | Composition |
|---|---|
| 3,4-dihydro-2H-1,4-benzoxazin-6-ol | A1 |
| 5-[(2-hydroxyethyl)amino]-2-methylphenol | A2 |
| 2-aminopyridin-3-ol | A3 |
| 3-amino-2-chloro-6-methylphenol | A4 |

Two series (S1) and (S2) of compositions (A1) to (A4) were therefore prepared.
Each of the compositions (A1) to (A4) of the series (S1) comprises 1-[3-*(2,5-diaminophenyl)propyl]-3-methyl-1H-imidazol-3-ium chloride dihydrochloride (example 1).
Each of the compositions (A1) to (A4) of the series (S2) comprises 4-[3-(2,5-diaminophenyl)propyl]-1,1-dimethylpiperazin-1-ium chloride dihydrochloride (example 2).

a) Procedure

Each composition (A1) to (A4) of each series (S1) and (S2) is applied to 1 g locks of natural Caucasian hair comprising 90% white hairs. After a leave-on time of 30 minutes at 27° C., the locks are rinsed, washed with a standard shampoo, rinsed again and then dried. The colorimetric data for each of the locks are then measured with a Minolta CM-3610d spectrophotometer.

b) Results

The following colorings were obtained:

Assessment of the dye evaluations of (S1) to (S2)

| Composition | S1 | S2 |
|---|---|---|
| A1 | ash yellow | light ash yellow |
| A2 | burgundy | mauve |
| A3 | brown | light brown |
| A4 | violet | violet |

### Example 2

### Evaluation of the Intensity and the Buildup: Invention vs. Comparative Composition

The comparative evaluation was carried out with the same dyeing vehicle as before, and according to the same operating conditions.

The oxidation bases are numbered as follows:
B1 is 4-[3-(2,5-diaminophenyl)propyl]-1,1-dimethylpiperazin-1-ium chloride dihydrochloride (Invention)
D is 2-[3-(4-methylpiperazin-1-yl)propyl]benzene-1,4-diamine tetrahydrochloride (Comparative composition)

The oxidation couplers are numbered as follows:
C1 is 3,4-dihydro-2H-1,4-benzoxazin-6-ol
C2 is 5-[(2-hydroxyethyl)amino]-2-methylphenol
C3 is 2-aminopyridin-3-ol
C4 is 3-amino-2-chloro-6-methylphenol

The colorimetric data for each of the locks are measured with a Minolta CM-3610d spectrophotometer. In this L*a*b* system, L* represents the lightness, a* indicates the green/red color axis and b* indicates the blue/yellow color axis.

The higher the value of L*, the lighter or less intense the color. Conversely, the lower the value of L*, the darker or more intense the color. The higher the value of a*, the redder the shade, and the higher the value of b*, the yellower the shade.

Evaluation of the Intensity L* of the Dyes

Bases

| | | D (comparative |
|---|---|---|
| Couplers | B1 (invention) | composition) |
| C1 | 49.94 | 56.11 |
| C2 | 45.48 | 53.99 |
| C3 | 46.86 | 57.99 |
| C4 | 35.80 | 44.62 |

The color buildup on hair corresponds to the variation in coloring between the locks of dyed NG hair (natural gray hair containing 90% white hairs) and the non-dyed (i.e. untreated) NG hair, which is measured by (AE) according to the following equation:

ΔE=\sqrt{(L*−Lₒ*)²+(a*−aₒ*)²+(b*−bₒ*)²}

In this equation, L*, a* and b* represent the values measured after dyeing of the NG hair, and L*, aₒ* and bₒ* represent the values measured before dyeing of the NG hair. The higher the ΔE value, the better the buildup of the coloring.

Evaluation of the Buildup ΔE of the Dyes

Bases

| | | D (comparative |
|---|---|---|
| Couplers | B1 (invention) | composition) |
| C1 | 10.36 | 4.95 |
| C2 | 20.72 | 10.41 |
| C3 | 15.75 | 3.84 |
| C4 | 34.20 | 22.81 |

It is apparent from the above results that the colorings obtained with the method of is the invention are significantly more intense and more colorfast than those obtained with the comparative composition.

### Example 3

### Evaluation of the Intensity and the Buildup: Invention vs. Another Comparative Composition

Dyeing vehicle:

| Ingredients | Amount | |
|---|---|---|
| 2-Octyldodecanol | 7.7 | g |
| Liquid petroleum jelly | 50 | g |
| Oxyethylenated (2 OE) lauryl alcohol | 2.0 | g |
| Oxyethylenated (4 OE) sorbitan monolaurate | 7.3 | g |
| Water | 33 | g |

Compositions:

| Compositions | Amount | |
|---|---|---|
| Oxidation base: para-phenylenediamine of | 0.05 | mmol |
| formula (I) (invention) or comparative | | |
| composition | | |
| Oxidation coupler | 0.05 | mmol |
| Oleogel vehicle | 1.0 | ml |
| 20% aqueous ammonia solution | drops | |
| 20-Volume aqueous hydrogen peroxide solution | 1.0 | ml |

The oxidation bases tested are the following:
B2 is 1-[3-(2,5-diaminophenyl)propyl]-3-methyl-1H-imidazol-3-ium chloride dihydrochloride (Invention)
D2 is 1-[2-(2,5-diaminophenyl)ethyl]-3-methyl-1 H-imidazol-3-ium chloride dihydrochloride (Comparative composition)

The oxidation couplers tested are the following:
C1 is 3,4-dihydro-2H-1,4-benzoxazin-6-ol
C5 is resorcinol
C6 is 2-methylbenzene-1,3-diol
C7 is 2-(2,4-diaminophenoxy)ethanol dihydrochloride
C8 is 1H-indol-6-ol

Each composition is applied to a 0.5 g lock of natural Caucasian hair comprising 90% white hairs. After a leave-on time of 30 minutes at ambient temperature, the locks are rinsed with water, washed with a standard shampoo, rinsed again and then dried. The colorimetric data for each of the locks are then measured with a Minolta CM-3610d spectrophotometer.

Evaluation of the Intensity L* of the Dyes

Ox. bases

| | | D2 (comparative |
|---|---|---|
| Couplers | B2 (invention) | composition) |
| C5 | 41.59 | 43.03 |
| C6 | 43.64 | 45.16 |
| C7 | 20.53 | 22.13 |
| C8 | 34.83 | 44.09 |

Evaluation of the Buildup ΔE of the Dyes

Ox. bases

| | | D2 (comparative |
|---|---|---|
| Couplers | B2 (invention) | composition) |
| C1 | 14.43 | 12.75 |
| C6 | 10.07 | 8.69 |
| C8 | 16.6 | 14.74 |

It is apparent from the above results that the dyeing method according to the invention which uses bases of cationic heterocyclic para-phenylenediamine type with a propylene chain vs a dyeing method which does not use a base of cationic heterocyclic para-phenylenediamine type with an ethylene chain, that the colorings obtained with the method of the invention are more intense than the comparative composition, and the buildup according to the invention is greater than that obtained with the comparative composition.

### Example 4

### Evaluation of the Intensity, the Buildup and the Shampoo Resistance of the Dyes vs. Other Comparative Composition

Composition (A) according to the present invention was prepared from the ingredients, the contents of which are indicated, as weight percentage of active material relative to the total weight of composition, in the table below:

| Ingredients | Composition A |
|---|---|
| 96° ethyl alcohol | 20.8 |
| 35% aqueous sodium metabisulfite solution | 0.23 |
| 40% aqueous solution of the pentasodium salt of | 0.48 |
| diethylenetriaminepentaacetic acid | |
| 60% aqueous solution of C₈-C₁₀ alkyl polyglucoside | 3.6 |
| Benzyl alcohol | 2.0 |
| Polyethylene glycol containing 8 ethylene oxide | 3.0 |
| units | |
| NH₄Cl | 4.32 |
| Aqueous ammonia containing 20% NH₃ | 2.94 |
| Compound of formula (I) | 10⁻³ mol |
| Oxidation coupler | 10⁻³ mol |
| Water | qs 100 |

At the time of use, each of the compositions is mixed with a 20-volume (6% by weight) aqueous hydrogen peroxide solution in a 1:1 ratio. The final pH of each of the mixtures is equal to 9.5. Each mixture thus obtained is applied to a lock of gray hair containing 90% white hairs. After a leave-on time of 30 minutes, the locks are rinsed, washed with a standard shampoo and then rinsed again. The locks are then dried.

The shampoo resistance of the color on the hair corresponds to the difference in color buildup before and after shampooing (˜50% DOP shampoo, 6 cycles of 1 shampoo wash +3 rinses, on a transparent Pall plate) or (˜2% DOP shampoo, 6 shampoo washes).

The light fastness of the color on the hair corresponds to the value of the difference in color buildup before and after exposure to light (Xenon-1600W, exposure 2 h 26 min, ˜1 month of normal life).

The compounds of formula (I) (oxidation bases) are numbered as follows:
B2 is 1-[3-(2,5-diaminophenyl)propyl]-3-methyl-1H-imidazol-3-ium chloride dihydrochloride (Invention)
B3 is 1-[3-(2,5-diaminophenyl)propyl]-2-methyl-1 H-pyrazol-2-ium chloride dihydrochloride (Invention)
B4 is 4-[3-(2,5-diaminophenyl)propyl]-4-methylthiomorpholin-4-ium chloride dihydrochloride (Invention)
D2′ is 2-[3-(1H-imidazol-1-yl)propyl]benzene-1,4-diamine chloride dihydrochloride (Comparative composition)
D3 is 2-[3-(1H-pyrazol-1-yl)propyl]benzene-1,4-diamine chloride dihydrochloride (Comparative composition)
D4 is 2-[3-(thiomorpholin-4-yl)propyl]benzene-1,4-diamine chloride dihydrochloride (Comparative composition)

The oxidation couplers are numbered as follows:
C2 is 5-[(2-hydroxyethyl)amino]-2-methylphenol
C3 is 2-aminopyridin-3-ol
C4 is 3-amino-2-chloro-6-methylphenol
C5 is resorcinol
C6 is 2-methylbenzene-1,3-diol
C8 is 1 H-indol-6-ol
C9 is 5-amino-2-methylphenol
010 is 2-(2,4-diaminophenoxy)ethanol hydrochloride

Evaluation of the Intensity of the Dyes: L*

Bases

| | | D2′ (comparative |
|---|---|---|
| Couplers | B2 (invention) | composition) |
| C2 | 28.05 | 31.04 |
| C4 | 23.30 | 26.66 |
| C6 | 31.8 | 34.7 |
| C9 | 24.43 | 30.57 |

It was observed visually that the keratin fibers treated with the cationic oxidation bases according to the invention make it possible to improve the intensity of the color of the fibers vs. the color obtained with the non-cationic comparative oxidation bases. This was corroborated with the results of the above table of L* measured which shows significant differences in improvement of the intensity for various couplers.

Evaluation of the Buildup of the Dyes ΔE

Bases

| | | D2′ (comparative |
|---|---|---|
| Couplers | B2 (invention) | composition) |
| C2 | 18.81 | 15.57 |
| C4 | 25.01 | 21.91 |
| C6 | 11.24 | 8.43 |
| C9 | 21.94 | 16.77 |

A much better color buildup was also observed for the cationic oxidation bases of the invention than for the comparative non-cationic oxidation bases. This observation is corroborated with the table of ΔE calculated from the values of L, a, b measured above.

Evaluation of the Shampoo Resistance of the Dyes

Bases

| | | D2′ comparative |
|---|---|---|
| Couplers | B2 invention | composition |
| C3 | 1.42 | 4.60 |
| C10 | 3.86 | 6.29 |

Bases

| | | D4 comparative |
|---|---|---|
| Couplers | B4 invention | composition |
| C3 | 1.75 | 4.57 |
| C5 | 0.95 | 2.26 |
| C8 | 1.38 | 3.19 |

It is apparent from the above results that the dyeing method according to the invention which uses bases of cationic heterocyclic para-phenylenediamine type vs. a dyeing method which uses a base of non-cationic heterocyclic para-phenylenediamine type, makes it possible to obtain colorations which are significantly more shampoo-resistant than that obtained with the comparative compositions.

1. -20. (canceled)

21. A compound chosen from compounds of formula (I) below: 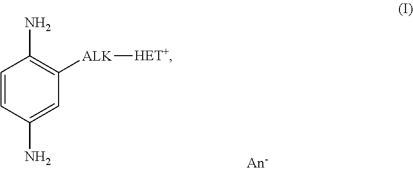
addition salts thereof with organic or inorganic acids or bases, optical isomers thereof, geometric isomers thereof, tautomers thereof, mesomers thereof, or solvates thereof: wherein:
ALK represents a linear or branched alkylene chain comprising from 3 to 8 carbon atoms which is optionally substituted;
HET⁺ represents a saturated or unsaturated, aromatic or non-aromatic cationic heterocyclic group, which is optionally substituted, comprising from 5 to 10 ring members, and comprising one or more ammonium groups; and
An⁻, which is present or absent, represents an inorganic or organic anionic counterion, ensuring the electrical neutrality of the molecule.

22. The compound of claim 21, wherein ALK represents a linear unsubstituted C₃-C₆ alkylene chain.

23. The compound of claim 21, wherein HET⁺ is an aromatic or non-aromatic, monocyclic or bicyclic, fused or non-fused heterocyclic group which contains one or more unsaturations, comprising from 5 to 10 ring members, and from 1 to 4 heteroatoms chosen from a nitrogen, oxygen, or sulfur atom; and further comprising one or more =N⁺(Rₑ)—; —N⁺(Rₑ)(R_{f})—; and/or —N⁺(Rₑ)(R_{f})(R_{g}) ammonium groups being borne by one or more substituents of the heterocycle or else said —N⁺(Rₑ)(R_{f})(R_{g}) ammonium group(s) being one or more substituents of the heterocycle, with Rₑ, R_{f}, R_{g} independently denoting a (C₁-C₆)alkyl radical or a hydroxy(C₁-C₆)alkyl radical; said cationic heterocyclic radical being chosen from:
i) cationic heterocycles, at least one of the ring members of which is an =N⁺(Rₑ)—, or —N⁺(Rₑ)(R_{f})— ammonium radical;
ii) heterocycles substituted by a radical bearing an —N⁺(Rₑ)(R_{f})(R_{g}) cationic group; and
ii) heterocycles substituted by an —N⁺(Rₑ)(R_{f})(R_{g}) cationic radical I.

24. The compound of claim 21, wherein HET⁺ is chosen from the formulae (A) to (P) below: 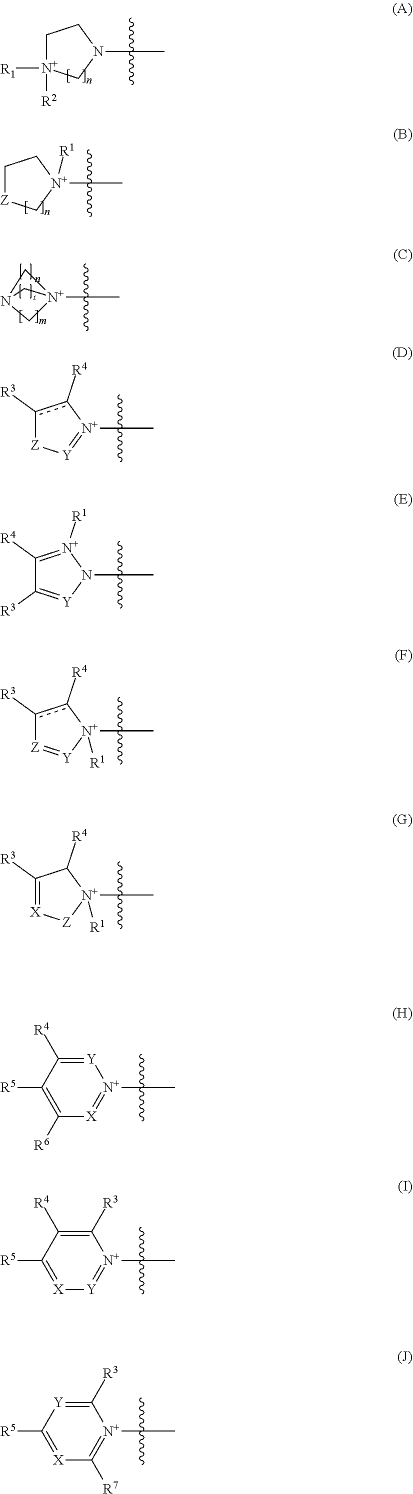 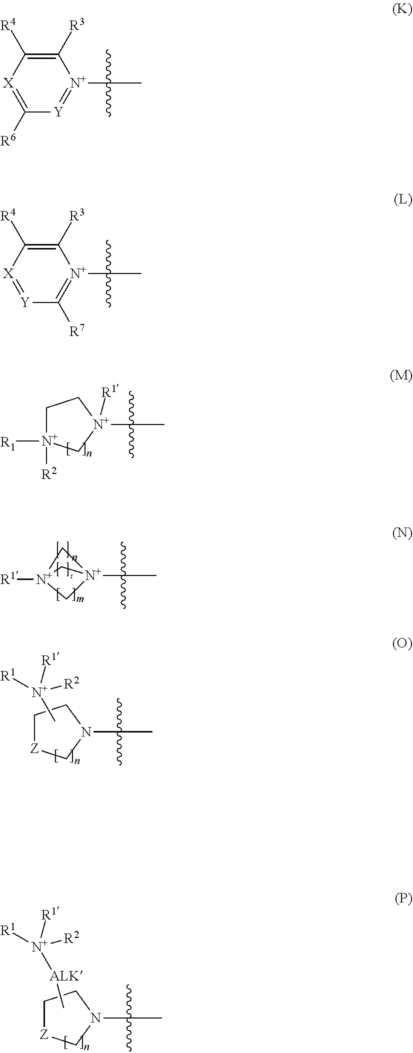
wherein
n being equal to 1, 2 or 3;
m and t, which are identical or different, being equal to 1, 2, or 3;
R¹, R¹′ and R², which are identical or different, represent a (C₁-C₆)alkyl group, or a hydroxy(C₁-C₆)alkyl group;
R³, R⁴, R⁵, R⁶ and R⁷, which are identical or different, represent a hydrogen atom, a halogen atom, or a group chosen from (C₁-C₆)alkyl, (C₁-C₆)alkoxy, (C₁-C₆)alkylthio, (di)(C₁-C₆)(alkyl)amino, nitro(so), R⁸—(Z′)ₚ—C(Z″)—(Z″′)_{q}— with p and q, which are identical or different, being equal to 0 or 1, Z′, Z″ and Z″′, which are identical or different, representing an oxygen atom, a sulfur atom or an N(R⁹) group with R⁸ and R⁹, which are identical or different, representing a hydrogen atom, a (C₁-C₄)alkyl group or a hydroxy(C₁-C₄)alkyl group; or two contiguous chosen from R³, R⁴, R⁵, R⁶, R⁸ and R⁹, form, together with the carbon atoms which bear them, a benzo group;
X and Y, which are identical or different, represent a nitrogen atom, or a group chosen from C(R⁸) with R⁸ or R⁸ represents an (C₁-C₄)alkylcarbonyl group;
Z representing an oxygen atom, a sulfur atom, a C(R⁸)(R⁹) group, or N(R⁹) group with R⁸ and R⁹;
ALK' represents a linear or branched (C₁-C₄)alkylene group, which is optionally substituted; 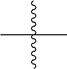
represents the part of the group linked to the rest of the molecule; and
- - - - - representing a single bond or double bond;
wherein for (O) and (P) the (R¹)(R′₁)(R²)N⁺— and (R¹)(R′₁)(R²)N⁺-ALK′-ammonium groups respectively are optionally borne by Z when Z represents a C(R⁸)(R⁹) group, in which case one of the two R⁸ or R⁹ groups is absent.

25. The compound of claim 21, wherein HET⁺ is chosen from (A) and (B) with n being equal to 1 or 2.

26. The compound of claim 23, wherein HET⁺ represents the (A₁) group: 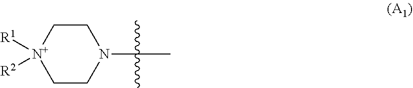
wherein R¹ and R², which are identical or different, representing a (C₁-C₄)alkyl group.

27. The compound of claim 24, wherein HET⁺ represents the group chosen from (B₁), (B₂), (B₃), (B₄), or (B₅) below: 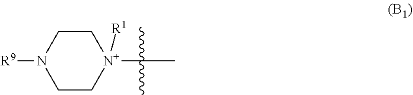
with R¹ and R⁹, which are identical or different, representing a (C₁-C₄)alkyl group; 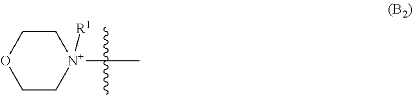
with R¹ representing a (C₁-C₄)alkyl group; 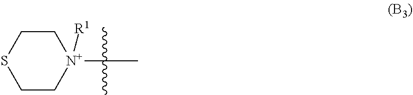
with R¹ representing a (C₁-C₄)alkyl group; 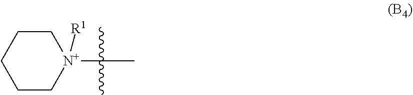
with R¹ representing a (C₁-C₄)alkyl group; 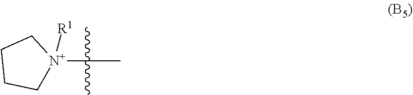
with R¹ representing a (C₁-C₄)alkyl group.

28. The compound of claim 24, wherein HET⁺ represents the (C₁) group below:

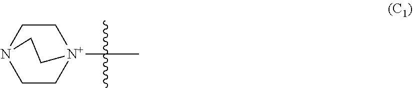 [SMILES: *[N+]12CCN(CC1)CC2]

29. The compound of claim 24, wherein HET⁺ represents the imidazolium group (D₁) or the mesomeric form thereof (D′1): 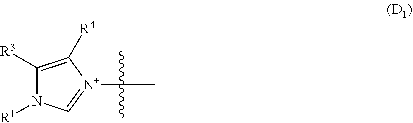
wherein R³ and R⁴ represent a hydrogen atom and R¹ represents a (C₁-C₄)alkyl group.

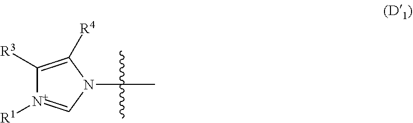

30. The compound of claim 24, wherein HET⁺ represents the pyrazolium group (E₁): 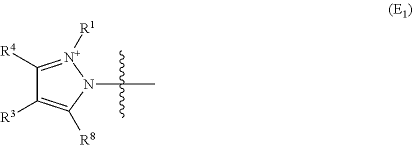
wherein R³, R⁴ and R⁸ represent a hydrogen atom and R¹ represents a (C₁-C₄)alkyl group.

31. The compound of claim 24, wherein HET⁺ represents the pyrrolium group (F₁): 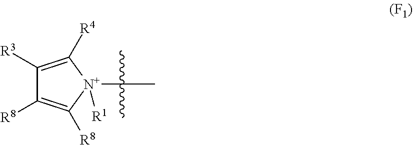
wherein R³, R⁴ and R⁸ represent a hydrogen atom and R¹ represents a (C₁-C₄)alkyl group.

32. The compound of claim 24, wherein HET⁺ represents the pyrrolinium group (O₁): 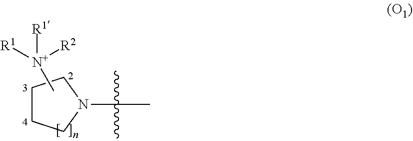
wherein R¹, R′¹, and R², represent a (C₁-C₄)alkyl group, n is equal to 1 or 2, wherein the (R¹)(R′₁)(R²)N⁺-ammonium group is in position 2, 3, or 4.

33. The compound of claim 24, wherein HET⁺ represents the pyrrolidinyl group (Pi): 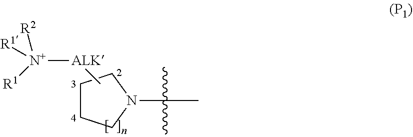
wherein R¹, R′¹, and R², represent a (C₁-C₄) group, n is equal to 1 or 2, ALK′ represents a linear (C₁-C₄)alkylene group, wherein the (R¹)(R′₁)(R²)N⁺-ALK-group is in position 2, 3, or 4.

34. The compound of claim 21, wherein the compound is chosen from compounds 1 to 13, inorganic or organic acid salts thereof, mesomers thereof, tautomers thereof, or solvates thereof:

| | | |
|---|---|---|
| Compound 1 | 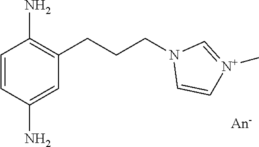 [SMILES: C[n+]1ccn(CCCc2cc(N)ccc2N)c1] | 1-[3-(2,5- diaminophenyl)propyl]-3- methyl-1H-imidazol-3- ium salt |
| Compound 2 | 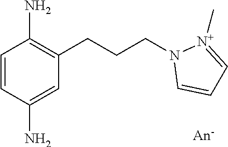 [SMILES: C[n+]1cccn1CCCc1cc(N)ccc1N] | 1-[3-(2,5- diaminophenyl)propyl]-2- methyl-1H-pyrazol-1-ium salt |
| Compound 3 | 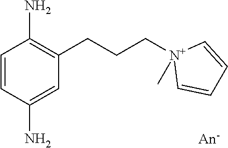 [SMILES: C[N+]1(CCCc2cc(N)ccc2N)C=CC=C1] | 1-[3-(2,5- diaminophenyl)propyl]-1- methyl-1H-pyrrol-1-ium salt |
| Compound 4 | 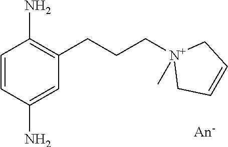 [SMILES: C[N+]1(CCCc2cc(N)ccc2N)CC=CC1] | 1-[3-(2,5- diaminophenyl)propyl]-1- methyl-2,5-dehydro-1H- pyrrol-2-ium salt |
| Compound 5 | 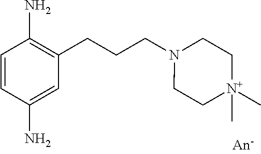 [SMILES: C[N+]1(C)CCN(CCCc2cc(N)ccc2N)CC1] | 4-[3-(2,5- diaminophenyl)propyl]- 1,1-dimethylpiperazin-1- ium salt |
| Compound 6 | 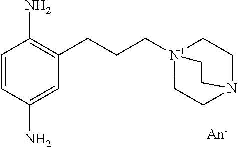 [SMILES: Nc1ccc(N)c(CCC[N+]23CCN(CC2)CC3)c1] | 1-[3-(2,5- diaminophenyl)propyl]-4- aza-1- azoniabicyclo[2.2.2]octane salt |
| Compound 7 | 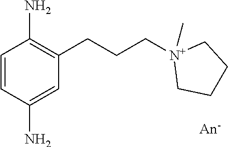 [SMILES: C[N+]1(CCCc2cc(N)ccc2N)CCCC1] | 1-[3-(2,5- diaminophenyl)propyl]-1- methylpyrrolidinium salt |
| Compound 8 | 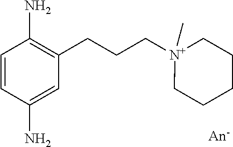 [SMILES: C[N+]1(CCCc2cc(N)ccc2N)CCCCC1] | 1-[3-(2,5- diaminophenyl)propyl]-1- methylpiperidinium salt |
| Compound 9 | 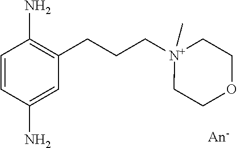 [SMILES: C[N+]1(CCCc2cc(N)ccc2N)CCOCC1] | 4-[3-(2,5- diaminophenyl)propyl]-4- methylmorpholin-4-ium salt |
| Compound 10 | 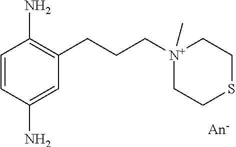 [SMILES: C[N+]1(CCCc2cc(N)ccc2N)CCSCC1] | 4-[3-(2,5- diaminophenyl)propyl]-4- methylthiomorpholin-4- ium salt |
| Compound 11 | 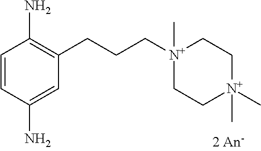 [SMILES: C[N+]1(C)CC[N+](C)(CCCc2cc(N)ccc2N)CC1] | 4-[3-(2,5- diaminophenyl)propyl]- 4,4-trimethylpiperazin-1- ium salt |
| Compound 12 | 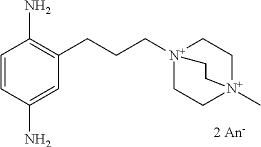 [SMILES: C[N+]12CC[N+](CCCc3cc(N)ccc3N)(CC1)CC2] | 1-[3-(2,5- diaminophenyl)propyl]-4- aza-1-methyl-1- azoniabicyclo[2.2.2]octane salt |
| Compound 13 | 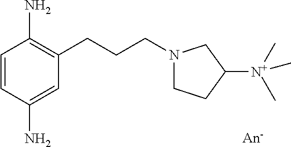 [SMILES: C[N+](C)(C)C1CCN(CCCc2cc(N)ccc2N)C1] | 1-[3-(2,5- diaminophenyl)propyl]-1- pyrrolidinyl-3- trimethylammonium salt |

wherein An⁻, which is identical or different, representing an anionic counterion.

35. A method of oxidation dyeing of keratin fibers comprising applying to the keratin fibers a compound chosen from compounds of formula (I) below: 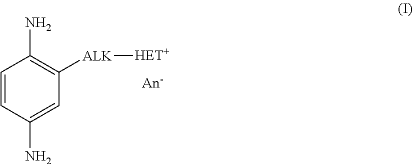
addition salts thereof with organic or inorganic acids or bases, optical isomers thereof, geometric isomers thereof, tautomers thereof, mesomers thereof, or solvates thereof:
wherein:
ALK represents a linear or branched alkylene chain comprising from 3 to 8 carbon atoms which is optionally substituted;
HET⁺ represents a saturated or unsaturated, aromatic or non-aromatic cationic heterocyclic group, which is optionally substituted, comprising from 5 to 10 ring members, and comprising one or more ammonium groups; and
An⁻, which is present or absent, represents an inorganic or organic anionic counterion, ensuring the electrical neutrality of the molecule.

36. A composition for dyeing keratin fibers, comprising, in a medium appropriate for dyeing, at least one compound of claim 21.

37. The composition of claim 36, wherein the composition further comprises one or more couplers chosen from meta-phenylenediamines, meta-aminophenols, meta-diphenols, naphthalene couplers, heterocyclic couplers, addition salts thereof, or mixtures thereof.

38. The composition of claim 37, wherein the composition further comprises at least one oxidizing agent chosen from hydrogen peroxide, urea peroxide, alkali metal bromates, persalts, peracids, or oxidase enzymes.

39. A method of dyeing keratin fibers, comprising applying to the keratin fibers the composition of claim 36.

40. A process for the preparation of a compound of formula (I) below: 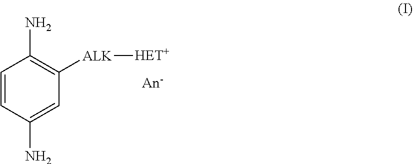
wherein:
ALK represents a linear or branched alkylene chain comprising from 3 to 8 carbon atoms which is optionally substituted;
HET⁺ represents a saturated or unsaturated, aromatic or non-aromatic cationic heterocyclic group, which is optionally substituted, comprising from 5 to 10 ring members, and comprising one or more ammonium groups; and
An⁻, which is present or absent, represents an inorganic or organic anionic counterion, ensuring the electrical neutrality of the molecule;
comprising: 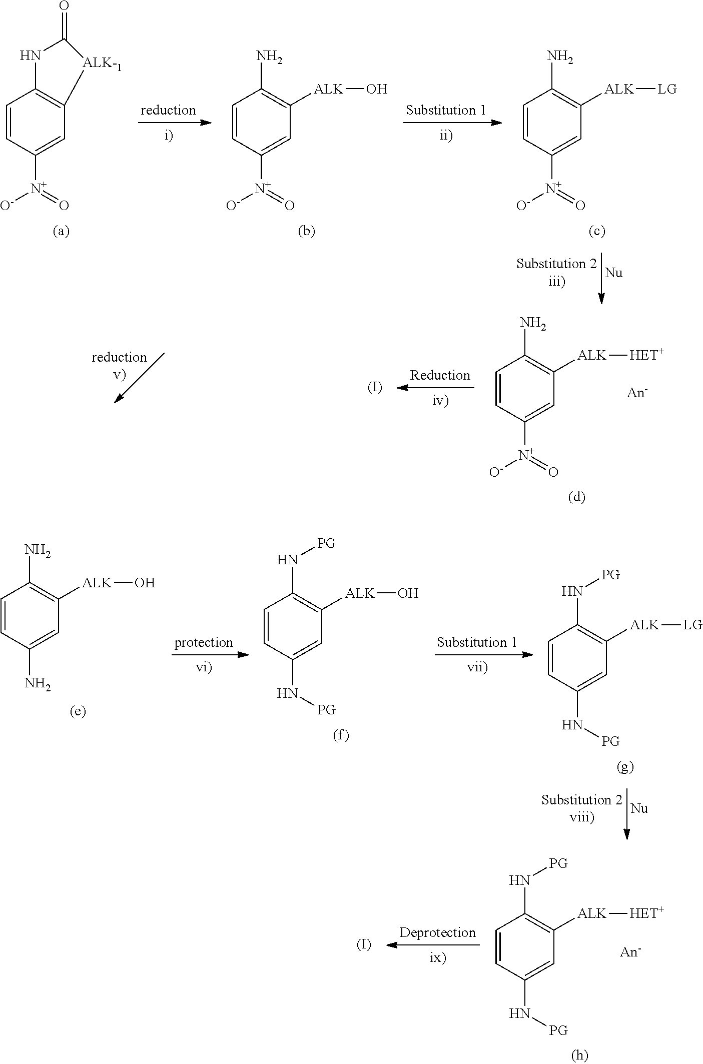
wherein Nu represents a Nucleophilic group comprising the cationic group HET⁺ , ALK₋₁ represents a linear or branched alkylene chain comprising from 2 to 7 carbon atoms which is optionally substituted, LG represents a leaving group, and PG represents a protecting group resistant to the reaction conditions throughout the synthesis up to the deprotection step;
which process consists:
either, in a first step i), in reducing the bicyclic amido derivative (a) in order to result in the 4-nitroaniline compound substituted in the a position with respect to the amino by a hydroxypropyl group (b); then, in a second step ii), in transforming the hydroxyl group of the compound (b) into a leaving group LG such as halogen, triflate, tosylate, mesylate, via a (C₁-C₆)alkylsulfonyl halide, step ii) performed in a polar or non-polar protic solvent; then, in a third step iii), in substituting the leaving group LG with a cationic group HET⁺ ; then, in a fourth step iv), in reducing the nitro compound (d) by catalytic hydrogenation, in order to result in the compound of formula (I);
or, in a second step v), in reducing the 4-nitroaniline derivative substituted in the a position with respect to the amino by a hydroxypropyl group (b), by catalytic hydrogenation, in order to result in the 1,4-phenylenediamine compound substituted in the a position with respect to the amino by a hydroxypropyl group (e), then, in a third step vi), in protecting the amino groups with a protecting group, via a reagent such as R—C(Yₐ)—Yₐ—C(Yₐ)—R′, with R and R′, which are identical or different, representing a (C₁-C₆)alkyl group and Ya, which is identical or different, representing an oxygen or sulfur atom; followed by steps vii) and viii) of substitutions 1 and 2 under the same conditions as during steps ii) and iii), in order to result in the compounds (c) and (d) respectively, and then the compound (h) is deprotected, in an organic solvent.
